(19) United States
(12) Patent Application Publication
(10) Pub. No.: US 2011/0182862 A1
(43) Pub. Date: Jul. 28, 2011

(54) ENDOPHYTIC FUNGUS AND USES THEREFOR

(76) Inventors: Wayne A. Green, Encinitas, CA (US); Markus J. Herrgard, San Diego, CA (US); Janne S. Kerovuo, San Diego, CA (US); David Lomelin, Encinitas, CA (US); Eric J. Mathur, Encinitas, CA (US); Toby H. Richardson, San Diego, CA (US); Ariel S. Schwartz, San Diego, CA (US); Gary A. Strobel, Bozeman, MT (US)

(21) Appl. No.: 12/753,760

(22) Filed: Apr. 2, 2010

Related U.S. Application Data
(60) Provisional application No. 61/166,681, filed on Apr. 3, 2009.
(60) Provisional application No. 61/230,648, filed on Jul. 31, 2009.

(51) Int. Cl. A01N 63/00; C12N 1/14; A01H 5/00; A01H 5/10; C12Q 1/02; C07H 21/00; C07K 2/00; C12N 1/15; A01P 5/00; A01P 1/00

(52) U.S. Cl. 424/93.5; 435/254.1; 800/298; 435/29; 536/23.1; 530/300; 435/254.11

(57) ABSTRACT

The present invention provides novel microorganisms, compositions and methods of use thereof, for treating, inhibiting or preventing the developing of a plant pathogenic disease and for killing or inhibiting growth of a variety of pests or pathogens. Provided are compositions comprising a novel endophytic fungal organism effective to inhibit the growth of or kill pests and pathogenic microbes, including Ganoderma boninense. Invention compositions are especially useful in preventing and treating basal stem rot in the oil palm, and can be applied on or in the vicinity of the plant or used to sterilize the plant growth medium prior to or concurrent with plant growth therein. The disclosure further provides substantially purified polynucleotides and polypeptides encoded thereby, together with methods of using those products, for example for making transgenic organism.

### CROSS REFERENCES TO RELATED APPLICATIONS

This nonprovisional application claims priority under 35 U.S.C. §119(e) to U.S. Patent Application No. 61/166,681 filed on Apr. 3, 2009 and U.S. Patent Application No. 61/230,648 filed on Jul. 31, 2009, the entire contents of which are hereby incorporated by reference.

### INCORPORATION OF THE SEQUENCE LISTING

The content of the following submission on compact discs is incorporated herein by reference in its entirety: A computer readable form (CRF) of the Sequence Listing on compact disc (file name: SGI1180-4_ST25.txt, date recorded: Apr. 2, 2010, size: 158,925 KB); a duplicate compact disc copy of the Sequence Listing (COPY 1) (file name: SGI1180-4_ST25.txt, date recorded: Apr. 2, 2010, size: 158,925 KB); and a duplicate compact disc copy of the Sequence Listing (COPY 2) (file name: SGI1180-4_ST25.txt, date recorded: Apr. 2, 2010, size: 158,925 KB).

### FIELD OF THE INVENTION

The present invention relates to the isolation and characterization of a novel endophytic fungal species that produces volatile organic compounds with biological activity against plant pathogens, particularly Ganoderma boninense, which is a causative agent of various plantation plant diseases, such as the oil palm disease Ganoderma Basal Stem Rot.

### BACKGROUND OF THE INVENTION

Oil palm, Elaeis guineensis, is the most important plantation crop in Malaysia. Four tons of palm oil are produced per hectare of cultivated palm trees on an annual basis. Many small private landowners are able to profit from the production and sale of the palm oil, making this an activity of great economic importance. Presently, Malaysia's oil palm industry is under threat as it is faced with a very serious plant disease problem. This problem is a prevailing and thus far incurable oil palm disease called Ganoderma Basal Stem Rot (BSR) caused by the fungus Ganoderma boninense. BSR is rapidly becoming the major threat to oil palm cultivation and palm oil production in Southeast Asia.

In BSR disease, basal stem rot is only one part of the disease cycle. Ganoderma boninense also causes a seedling disease and an upper stem rot of more developed palms. An understanding of spore dispersal of this pathogen provides an insight into the multiple roles of G. boninense spores in the infection process, leading to the three distinct phases of this important plant disease. This pathogenic organism is also prevalent on other major plantation plants including coconut, betel nut, tea, cacao, acacia and poplar.

With no effective cure at present, BSR has a huge economic impact to the Malaysian oil palm industry. Thus, plant health is crucial in obtaining maximal productivity of the oil palm and techniques, methods and management ideas are needed to control BSR. Attempts to control this disease with agrochemicals have not been very successful. This could be due to the fact that the oil palms already possessed latent fungal infections at the time of the chemical treatment. Biological control agents have also been tried against Ganoderma with limited success. Saprophytic organisms (such as Trichoderma harzianum) merely arrest the spread of disease by competing against Ganoderma to reduce its opportunity to colonize oil palm roots.

BSR is a particular concern because the activity of replanting oil palms can accelerate spread of the disease. It is well known that successive replanting of oil palms can be rapidly exploited by soil borne fungi such as Ganoderma. Soils that continuously support the growth of palms eventually act as a reservoir for Ganoderma fruiting structures and spores of this organism. Soils that are replanted with new palms are immediately exposed to a high load of inoculum (spores) and eventually become infected by Ganoderma boninense.

It is widely believed that the problem of basal stem rot of palm will become more serious later in the 21st century as more and more established plantations become due for second or even third replanting. Environmental considerations, coupled with governmental directives, will reduce exploitation of new forest areas, making further replanting of these crops inevitable. There is a need for integrated management systems for Ganoderma and related diseases to maintain the success of the oil palm industry.

An endophyte is an endosymbiont, often a bacterium or fungus, that lives within a plant for at least part of its life without causing apparent disease. Endophytes are ubiquitous and have been found in all the species of plants studied to date Endophytes may be transmitted either vertically (directly from parent to offspring) or horizontally (from individual to unrelated individual). Vertically transmitted fungal endophytes are typically asexual and transmit from the maternal plant to offspring via fungal hyphae penetrating the host's seeds. Since their reproductive fitness is intimately tied to that of their host plant, these fungi are often mutualistic. Conversely, horizontally transmitted fungal endophytes are sexual and transmit via spores that can be spread by wind and/or insect vectors. Endophytes can benefit host plants by preventing pathogenic organisms from colonizing them. Extensive colonization of the plant tissue by endophytes creates a “barrier effect”, where the local endophytes outcompete and prevent pathogenic organisms from taking hold. Endophytes may also produce chemicals which inhibit the growth of competitors, including pathogenic organisms.

Various endophytes, particularly fungi, have been used in order to manage plant diseases by targeting the growth and viability of plant pathogens. In the case of BSR disease, the use of endophytes would also be preferred to other biological control agents as they are internal colonizers, with better ability to compete within the vascular systems, limiting Ganoderma for both nutrients and space during its proliferation. Fungi of the Muscodor genus have been successfully used to target other types of plant pathogens. There are a variety of Muscodor species, which all share the same common features of growing slowly, having a felt-like mycelium, having a distinctive odor, and having no detrimental effects on higher plants. However, there are cultural, chemical, and molecular differences between them. For instance, naphthalene and azulene derivatives are produced by Muscodor albus while Muscodor vitigenus produces only naphthalene as the most prominent volatile substance (Strobel et al., 2001, Microbiology 147: 2943-2950 and Daisy et al., 2002, Microbiology 148: 3737-3741).

The novel Muscodor species of the present invention, with unusual biochemical and biological properties, can be distinguished by the profile of the volatile organic compounds it produces, and its unparallel ability to kill plant pathogens such as Ganoderma. The Muscodor strobelii strains provided herein can be completely lethal to Ganoderma boninense. No other prior isolates of Muscodor spp. produce the same volatile organic compounds, or are capable of completely killing Ganoderma. Thus, this novel endophyte species satisfies the stated needs for effective Ganoderma control both in soil and in plants, and provides related advantages as well.

### SUMMARY OF THE INVENTION

In one aspect, the invention provides an isolated culture, or a biologically pure culture, of a strain of Muscodor strobelii. One embodiment of this aspect provides a strain deposited at the Agricultural Research Service Culture Collection and having accession number NRRL 50288. In certain preferred embodiments, the culture of strain of Muscodor strobelii is capable of producing isobutyric acid or a derivative thereof, such as isobutyric anhydride or isobutyric acid, methyl ester, optionally in combination with one or more aristolene, bergamotene, caryophyllene, gurjunene, isolongifolene, patchoulene, or a derivative of any thereof.

In some preferred embodiments, the isolated culture of Muscodor strobelii further comprises an agriculturally effective amount of a pesticidal compound or composition. The additional compound or composition may be an acaricide, a bactericide, a fungicide, an insecticide, a microbicide, a nematicide, a fertilizer, or a food preservative.

The composition in some embodiments may be in the form of a powder, a granule, a pellet, a gel, an aqueous suspension, a solution or an emulsion. The composition may be provided with a carrier. The carrier can be a seed.

Another aspect of the invention provides a method for treating, inhibiting or preventing the development of a plant pathogenic disease. The method involves applying an isolated culture of Muscodor strobelii on or in the vicinity of a host plant. In a preferred embodiment, the pathogen causing the pathogenic disease may be Aspergillus fumigatus, Botrytis cinerea, Cerpospora betae, Curvularia sp., Ganoderma boninense, Geotrichum candidum, Mycosphaerella fijiensis, Phytophthora palmivora, Phytophthora ramorum, Pythium ultimum, Rhizoctonia solani, Rhizopus sp., Schizophyllum sp., Sclerotinia sclerotiorum, Verticillium dahliae, or Xanthomonas axonopodis. In another preferred embodiment, the host plant is susceptible to disease caused by Ganoderma boninense. In another preferred embodiment, the host plant is an oil plam plant. In certain embodiments, the Muscodor strobelii is established as an endophyte on the plant.

Another further aspect of the invention provides a non-naturally occurring oil palm cultivar that is an oil palm infected with an isolated culture of Muscodor strobelii. In some embodiments, the disclosure further provides seed, reproductive tissue, vegetative tissue, plant parts, and progeny of the non-naturally occurring oil palm cultivar. In other embodiments, the present disclosure also provides products comprising material derived from a non-naturally occurring cultivar.

In another aspect of the invention, there are provided methods for treating, inhibiting or preventing a plant pathogen-related disease. In certain embodiments, the methods involve growing a culture of Muscodor strobelii in the vicinity of the host plant, or in the growth medium or soil of the host plant prior to or concurrent with plant growth in the growth medium or soil. In one or more of the above embodiments, the method is effective to kill the plant pathogen. Thus, the invention provides a method of killing a plant pathogen comprising growing a culture of Muscodor strobelii in the vicinity of the plant pathogen. In various embodiments, the plant pathogen is associated with the host plant, or is in the growth medium or soil of the host plant prior to or concurrent with plant growth in the growth medium or soil.

Another aspect of the invention provides a method for screening microbial strains that can be useful for treating, inhibiting or preventing the development of a plant pathogenic disease. The method involves (i) co-culturing a Muscodor strobelii strain with one or more candidate microbial strains, (ii) selecting one or more viable microbial strains after the co-culturing, and (iii) characterizing the one or more selected microbial strains. As described above, the screening method is used to identify one or more microbial strains useful for treating, inhibiting or preventing the development of a plant pathogenic disease. The present invention further includes the microbial strains obtained from the method as described above.

Another further aspect of the invention relates to a method for killing, inhibiting or preventing the development of an undesired organism, such as a fungus, a bacterium, a microorganism, a nematode, and an insect. The method involves exposing or contacting the organism to or with an effective amount of the invention composition.

In another further aspect of the invention, the present disclosure provides substantially purified nucleic acid molecules and the polypeptides encoded by such molecules from Muscodor strobelii. Polynucleotide and polypeptide sequences from Muscodor strobelii of the present disclosure are provided in the attached Sequence Listing.

The present disclosure also provides nucleotide sequences that hybridize under high stringency conditions to any one of the nucleotide sequences in the Sequence Listing, complements of nucleotide sequences that hybridize under high stringency conditions to any of the nucleotide sequences in the Sequence Listing, and fragments of either. The disclosure also provides nucleotides exhibiting a 70% or greater identity to any one of the nucleotide sequences in the Sequence Listing, complements of the nucleotide sequences exhibiting a 70% or greater identity to any one of the nucleotide sequences in the Sequence Listing, and fragments of either. The disclosure further provides nucleotide sequences encoding polypeptides that exhibit a 50% or greater identity to any one of the polypeptides in the Sequence Listing.

The disclosure also provides nucleotide sequences that are an interfering RNA to any one of the nucleotide sequences from Muscodor strobelii in the Sequence Listing; nucleotide sequences that are an interfering RNA to nucleotide sequences hybridizing under high stringency conditions to any one of the nucleotide sequences in the Sequence Listing; nucleotide sequences that are an interfering RNA to complements of nucleotide sequences hybridizing under high stringency conditions to any of the nucleotide sequences in the Sequence Listing; and nucleotides that are an interfering RNA to fragments of nucleotide sequences hybridizing under high stringency conditions to any one of the nucleotide sequences in the Sequence Listing or complements of nucleotide sequences hybridizing under high stringency conditions to any of the nucleotide sequences in the Sequence Listing.

The disclosure further provides nucleotide sequences that are an interfering RNA to nucleotides exhibiting a 70% or greater identity to any one of the nucleotide sequences in the Sequence Listing; nucleotides sequences that are an interfering RNA to complements of the nucleotide sequences exhibiting a 70% or greater identity to any one of the nucleotide sequences in the Sequence Listing; and nucleotides that are an interfering RNA to fragments of nucleotides exhibiting a 70% or greater identity to any one of the nucleotide sequences in the Sequence Listing or complements of the nucleotide sequences exhibiting a 70% or greater identity to any one of the nucleotide sequences in the Sequence Listing.

The disclosure also provides nucleotide sequences that are an interfering RNA to nucleotide sequences encoding polypeptides that exhibit a 50% or greater identity to any one of the polypeptides in the Sequence Listing.

The disclosure further provides substantially purified polypeptides, the peptides encoded by nucleic acid molecules including nucleic acids that hybridize under high stringency conditions to any one of the nucleotide sequences in the Sequence Listing, nucleic acids that are a complement of nucleotide sequences hybridizing under high stringency conditions to any one of the nucleotide sequences in the Sequence Listing, and nucleic acids comprising a fragment of a nucleotide sequence hybridizing under high stringency conditions to any one of the nucleotide sequences in the Sequence Listing or a complement of a nucleotide sequence hybridizing under high stringency conditions to any one of the nucleotide sequences in the Sequence Listing.

The disclosure also provides substantially purified polypeptides, the peptides encoded by nucleic acid molecules including nucleic acids with a nucleotide sequence exhibiting a 70% or greater identity to any one of the nucleotide sequences in the Sequence Listing; nucleic acids that are a complement of a nucleotide sequence exhibiting a 70% or greater identity to any one of the nucleotide sequences in the Sequence Listing; and nucleic acids comprising a fragment of a nucleotide sequence exhibiting a 70% or greater identity to any one of the nucleotide sequences in the Sequence Listing or a complement of a nucleotide sequence exhibiting a 70% or greater identity to any one of the nucleotide sequences in the Sequence Listing.

The disclosure further provides substantially purified polypeptides, the peptides encoded by nucleotide sequences encoding an amino acid sequence that exhibits a 50% or greater identity to any one of the polypeptides in the Sequence Listing.

The present disclosure further provides transformed cells including a first nucleic acid molecule corresponding to any of the nucleotide sequences from Muscodor strobelii in the Sequence Listing; a nucleotide sequence hybridizing under high stringency conditions to any one of the nucleotide sequences in the Sequence Listing, a complement thereof or a fragment of either; a nucleotide sequence exhibiting a 70% or greater identity to any one of the nucleotide sequences in the Sequence Listing, a complement thereof or a fragment of either; a nucleotide sequence encoding an amino acid sequence that exhibits a 50% or greater identity to any one of the polypeptides in the Sequence Listing; or a nucleotide sequence that is an interfering RNA to any one of the nucleotide sequences from Muscodor strobelii in the Sequence Listing, to nucleotide sequences hybridizing under high stringency conditions to any one of the nucleotide sequences in the Sequence Listing, to complements of nucleotide sequences hybridizing under high stringency conditions to any of the nucleotide sequences in the Sequence Listing, to fragments of nucleotide sequences hybridizing under high stringency conditions to any one of the nucleotide sequences in the Sequence Listing or complements of nucleotide sequences hybridizing under high stringency conditions to any of the nucleotide sequences in the Sequence Listing, to nucleotides exhibiting a 70% or greater identity to any one of the nucleotide sequences in the Sequence Listing, to complements of the nucleotide sequences exhibiting a 70% or greater identity to any one of the nucleotide sequences in the Sequence Listing, to fragments of nucleotides exhibiting a 70% or greater identity to any one of the nucleotide sequences in the Sequence Listing or complements of the nucleotide sequences exhibiting a 70% or greater identity to any one of the nucleotide sequences in the Sequence Listing, and to nucleotide sequences encoding polypeptides that exhibit a 50% or greater identity to any one of the polypeptides in the Sequence Listing. The first nucleic acid molecule is operably linked to a second nucleic acid molecule, which is heterologous with respect to the first nucleic acid molecule.

The disclosure also provides transgenic organisms with transformed cells including a first nucleic acid molecule corresponding to any of the nucleotide sequences from Muscodor strobelii in the Sequence Listing; a nucleotide sequence hybridizing under high stringency conditions to any one of the nucleotide sequences in the Sequence Listing, a complement thereof or a fragment of either; a nucleotide sequence exhibiting a 70% or greater identity to any one of the nucleotide sequences in the Sequence Listing, a complement thereof or a fragment of either; a nucleotide sequence encoding an amino acid sequence that exhibits a 50% or greater identity to any one of the polypeptides in the Sequence Listing; or a nucleotide sequence that is an interfering RNA to any one of the nucleotide sequences from Muscodor strobelii in the Sequence Listing, to nucleotide sequences hybridizing under high stringency conditions to any one of the nucleotide sequences in the Sequence Listing, to complements of nucleotide sequences hybridizing under high stringency conditions to any of the nucleotide sequences in the Sequence Listing, to fragments of nucleotide sequences hybridizing under high stringency conditions to any one of the nucleotide sequences in the Sequence Listing or complements of nucleotide sequences hybridizing under high stringency conditions to any of the nucleotide sequences in the Sequence Listing, to nucleotides exhibiting a 70% or greater identity to any one of the nucleotide sequences in the Sequence Listing, to complements of the nucleotide sequences exhibiting a 70% or greater identity to any one of the nucleotide sequences in the Sequence Listing, to fragments of nucleotides exhibiting a 70% or greater identity to any one of the nucleotide sequences in the Sequence Listing or complements of the nucleotide sequences exhibiting a 70% or greater identity to any one of the nucleotide sequences in the Sequence Listing, and to nucleotide sequences encoding polypeptides that exhibit a 50% or greater identity to any one of the polypeptides in the Sequence Listing. The first nucleic acid molecule is operably linked to a second nucleic acid molecule, which is heterologous with respect to the first nucleic acid molecule. In some preferred embodiments, the transgenic organism is an endophyte or a plant.

These and other objects and features of the invention will become more fully apparent when the following detailed description of the invention is read in conjunction with the accompanying drawings.

### BRIEF DESCRIPTION OF THE DRAWINGS

FIG. 1 shows a scanning electron micrograph of Muscodor strobelii, magnified at 300×.

FIG. 2 shows an infection site of Ganoderma boninense as it is attacking the rootlet of oil palm in vitro.

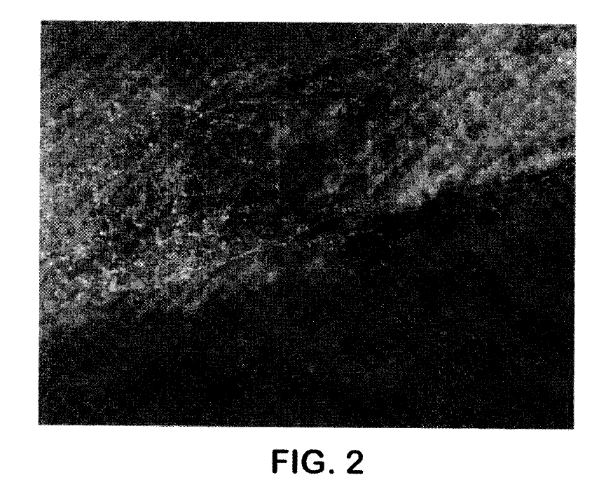

FIG. 3 shows GC/MS data of the SPME fiber analysis of the four samples in the soil treatment experiment described in Example 4. The compound eluting at 4.45 min is equivalent to isobutyric acid and is found only in samples containing Muscodor strobelii.

FIG. 4 shows a mixture of empty fruit bunches (EFBs) and rice used to support the growth of Muscodor strobelii. This mixture when dried can be used to treat soil and plant parts to eliminate pathogens.

### DETAILED DESCRIPTION OF THE INVENTION

### Definitions

Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

The singular form “a”, “an”, and “the” include plural references unless the context clearly dictates otherwise. For example, the term “a cell” includes one or more cells, including mixtures thereof.

Amino acid: As used herein, the term “amino acid” refers to naturally occurring and synthetic amino acids, as well as amino acid analogs and amino acid mimetics that function in a manner similar to the naturally occurring amino acids. Naturally occurring amino acids are those encoded by the genetic code, including D/L optical isomers, as well as those amino acids that are later modified, e.g., hydroxyproline, y-carboxyglutamate, and O-phosphoserine. Amino acid analogs refer to compounds that have the same basic chemical structure as a naturally occurring amino acid, i.e., a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refer to chemical compounds that have a structure that is different from the general chemical structure of an amino acid, but that functions in a manner similar to a naturally occurring amino acid.

Antibiotic: The term “antibiotic”, as used herein, refers to any substance that is able to kill or inhibit the growth of a microorganism. Antibiotics may be produced by any one or more of the following: 1) a microorganism, 2) a synthetic process, or 3) a semisynthetic process. An antibiotic may be a microorganism that secretes a volatile organic compound. Furthermore, an antibiotic may be a volatile organic compound secreted by a microorganism.

Bactericidal: The term “bactericidal, as used herein, refers to the ability of a substance to increase mortality or inhibit the growth rate of bacteria.

Biological control: As used herein, “biological control” is defined as control of a pathogen or insect or any other undesirable organism by the use of a second organism. An example of a known mechanism of biological control is the use of microorganisms that control root rot by out-competing fungi for space on the surface of the root, or microorganisms that either inhibit the growth of or kill the pathogen. The “host plant” in the context of biological control is the plant that is susceptible to disease caused by the pathogen. In the context of isolation of an organism, such as a fungal species, from its natural environment, the “host plant” is a plant that supports the growth of the fungus, for example, a plant of a species the fungus is an endophyte of.

Composition: A “composition” is intended to mean a combination of active agent and another compound, carrier or composition, inert (for example, a detectable agent or label or liquid carrier) or active, such as a pesticide.

Culturing: The term ‘culturing’, as used herein, refers to the propagation of organisms on or in media of various kinds.

Derivative: As used herein, a “derivative” of a chemical compound is a compound that can be chemically or biologically derived from the original compound, for example by the addition, substitution or deletion of chemical components of the original compound. For example, a derivative may be an isomer of the referenced compound, an anhydride of the referenced compound, or has one or more chemical groups added or substituted with respect to the referenced compound. For example, propanoic acid, 2-methyl, 3-methylbutyl ester is considered to be a derivative of propanoic acid.

Domain: As used herein, “Domains” are groups of substantially contiguous amino acids in a polypeptide that can be used to characterize protein families and/or parts of proteins. Such domains have a “fingerprint” or “signature” that can comprise conserved primary sequence, secondary structure, and/or three-dimensional conformation. Generally, domains are correlated with specific in vitro and/or in vivo activities. A domain can have a length of from 10 amino acids to 400 amino acids, e.g., 10 to 50 amino acids, or 25 to 100 amino acids, or 35 to 65 amino acids, or 35 to 55 amino acids, or 45 to 60 amino acids, or 200 to 300 amino acids, or 300 to 400 amino acids.

Effective amount: An “effective amount”, as used herein, is an amount sufficient to affect beneficial or desired results. An effective amount can be administered in one or more administrations. In terms of treatment, inhibition or protection, an effective amount is that amount sufficient to ameliorate, stabilize, reverse, slow or delay progression of the target infection or disease states.

Endogenous: The term “endogenous”, as used herein, refers to any component, such as a polynucleotide, polypeptide or protein sequence, which is a natural part of a cell or organism regenerated from said cell.

Exogenous: “Exogenous” with respect to a nucleic acid indicates that the nucleic acid is part of a recombinant nucleic acid construct, or is not in its natural environment. For example, an exogenous nucleic acid can be a sequence from one species introduced into another species, i.e., a heterologous nucleic acid. Typically, such an exogenous nucleic acid is introduced into the other species via a recombinant nucleic acid construct. An exogenous nucleic acid can also be a sequence that is native to an organism and that has been reintroduced into cells of that organism. An exogenous nucleic acid that includes a native sequence can often be distinguished from the naturally occurring sequence by the presence of non-natural sequences linked to the exogenous nucleic acid, e.g., non-native regulatory sequences flanking a native sequence in a recombinant nucleic acid construct. In addition, stably transformed exogenous nucleic acids typically are integrated at positions other than the position where the native sequence is found. It will be appreciated that an exogenous nucleic acid may have been introduced into a progenitor, and not into the cell under consideration. For example, a transgenic organism containing an exogenous nucleic acid can be the progeny of a sexual cross or matting between a stably transformed organism and a non-transgenic organism. Such progeny are considered to contain the exogenous nucleic acid.

Expression: As used herein, “expression” refers to the process of converting genetic information of a polynucleotide into RNA through transcription, which is catalyzed by an enzyme, RNA polymerase, and into protein, through translation of mRNA on ribosomes.

Functionally Comparable Protein: The phrase “functionally comparable protein”, as used herein, describes those proteins that have at least one characteristic in common. Such characteristics include sequence similarity, biochemical activity, transcriptional pattern similarity and phenotypic activity. Typically, the functionally comparable proteins share some sequence similarity or at least one biochemical. Within this definition, homologs, orthologs, paralogs and analogs are considered to be functionally comparable. In addition, functionally comparable proteins generally share at least one biochemical and/or phenotypic activity.

Functionally comparable proteins will give rise to the same characteristic to a similar, but not necessarily the same, degree. Typically, functionally comparable proteins give the same characteristics where the quantitative measurement due to one of the comparables is at least 20% of the other; more typically, between 30 to 40%; more typically, between 50-60%; even more typically, between 70 to 80%; even more typically, between 90 to 95%; even more typically, between 98 to 100% of the other.

Fungicidal: As used herein, “fungicidal” refers to the ability of a substance to decrease the rate of growth of fungi or to increase the mortality of fungi.

Fungus: The term “fungus” or “fungi”, as used herein, includes a wide variety of nucleated spore-bearing organisms that are devoid of chlorophyll. Examples of fungi include yeasts, molds, mildews, rusts, and mushrooms.

Heterologous polypeptides: A “Heterologous polypeptide”, as used herein, refers to a polypeptide that is not a naturally occurring polypeptide in a cell, e.g., a transgenic plant cell transformed with and expressing the coding sequence for a nitrogen transporter from a Muscodor species.

Heterologous sequences: “Heterologous sequences”, as used herein, are those that are not operatively linked or are not contiguous to each other in nature. For example, a promoter from oil palm is considered heterologous to a Muscodor strobelii coding region sequence. Also, a promoter from a gene encoding a growth factor from oil palm is considered heterologous to a sequence encoding the oil palm receptor for the growth factor. Regulatory element sequences, such as UTRs or 3′ end termination sequences that do not originate in nature from the same gene as the coding sequence, are considered heterologous to said coding sequence. Elements operatively linked in nature and contiguous to each other are not heterologous to each other. On the other hand, these same elements remain operatively linked but become heterologous if other filler sequence is placed between them. Thus, the promoter and coding sequences of an oil palm gene expressing an amino acid transporter are not heterologous to each other, but the promoter and coding sequence of an oil palm gene operatively linked in a novel manner are heterologous.

Isolated nucleic acid: An “isolated nucleic acid”, as used herein, includes a naturally-occurring nucleic acid, provided one or both of the sequences immediately flanking that nucleic acid in its naturally-occurring genome is removed or absent. Thus, an isolated nucleic acid includes, without limitation, a nucleic acid that exists as a purified molecule or a nucleic acid molecule that is incorporated into a vector or a virus. A nucleic acid existing among hundreds to millions of other nucleic acids within, for example, cDNA libraries, genomic libraries, or gel slices containing a genomic DNA restriction digest, is not to be considered an isolated nucleic acid.

Modulation: As used herein, “modulation” of the level of a compound or constituent refers to the change in the level of the indicated compound or constituent that is observed as a result of expression of, or transcription from, an exogenous nucleic acid in an endophyte cell or in a plant cell. The change in level is measured relative to the corresponding level in control endophytes or plants.

Mutant: As used herein, the term “mutant” or “variant” refers to a modification of the parental strain in which the desired biological activity is similar to that expressed by the parental strain. For example, in the case of Muscodor the “parental strain” is defined herein as the original Muscodor strain before mutagenesis. Mutants or variants may occur in nature without the intervention of man. They also are obtainable by treatment with or by a variety of methods and compositions known to those of skill in the art. For example, a parental strain may be treated with a chemical such as N-methyl-N′-nitro-N-nitrosoguanidine, ethylmethanesulfone, or by irradiation using gamma, x-ray, or UV-irradiation, or by other means well known to those practiced in the art.

Nematicidal: The term “nematicidal”, as used herein, refers to the ability of a substance to increase mortality or inhibit the growth rate of nematodes.

Nucleic acid and polynucleotide: The terms “Nucleic acid” and “polynucleotide” are used interchangeably herein, and refer to both RNA and DNA, including cDNA, genomic DNA, synthetic DNA, and DNA or RNA containing nucleic acid analogs. Polynucleotides can have any three-dimensional structure. A nucleic acid can be double-stranded or single-stranded (i.e., a sense strand or an antisense strand). Non-limiting examples of polynucleotides include genes, gene fragments, exons, introns, messenger RNA (mRNA), transfer RNA, ribosomal RNA, siRNA, micro-RNA, ribozymes, cDNA, recombinant polynucleotides, branched polynucleotides, nucleic acid probes and nucleic acid primers. A polynucleotide may contain unconventional or modified nucleotides.

Operably linked: As used herein, “operably linked” refers to the positioning of a regulatory region and a sequence to be transcribed in a nucleic acid so that the regulatory region is effective for regulating transcription or translation of the sequence. For example, to operably link a coding sequence and a regulatory region, the translation initiation site of the translational reading frame of the coding sequence is typically positioned between one and about fifty nucleotides downstream of the regulatory region. A regulatory region can, however, be positioned as much as about 5,000 nucleotides upstream of the translation initiation site, or about 2,000 nucleotides upstream of the transcription start site.

Percentage of percent identity: “Percentage of sequence identity”, as used herein, is determined by comparing two optimally locally aligned sequences over a comparison window defined by the length of the local alignment between the two sequences. The amino acid sequence in the comparison window may comprise additions or deletions (e.g., gaps or overhangs) as compared to the reference sequence (which does not comprise additions or deletions) for optimal alignment of the two sequences. Local alignment between two sequences only includes segments of each sequence that are deemed to be sufficiently similar according to a criterion that depends on the algorithm used to perform the alignment (e.g. BLAST). The percentage identity is calculated by determining the number of positions at which the identical nucleic acid base or amino acid residue occurs in both sequences to yield the number of matched positions, dividing the number of matched positions by the total number of positions in the window of comparison and multiplying the result by 100. Optimal alignment of sequences for comparison may be conducted by the local homology algorithm of Smith and Waterman (1981) Add. APL. Math. 2:482, by the global homology alignment algorithm of Needleman and Wunsch (1970) J Mol. Biol. 48:443), by the search for similarity method of Pearson and Lipman (1988) Proc. Natl. Acad. Sci. (USA) 85: 2444, by heuristic implementations of these algorithms (NCBI BLAST, WU-BLAST, BLAT, SIM, BLASTZ), or by inspection. Given that two sequences have been identified for comparison, GAP and BESTFIT are preferably employed to determine their optimal alignment. Typically, the default values of 5.00 for gap weight and 0.30 for gap weight length are used. The term “substantial sequence identity” between polynucleotide or polypeptide sequences refers to polynucleotide or polypeptide comprising a sequence that has at least 50% sequence identity, preferably at least 70%, preferably at least 80%, more preferably at least 85%, more preferably at least 90%, even more preferably at least 95%, and most preferably at least 96%, 97%, 98% or 99% sequence identity compared to a reference sequence using the programs.

Query nucleic acid and amino acid sequences can be searched against subject nucleic acid or amino acid sequences residing in public or proprietary databases. Such searches can be done using the National Center for Biotechnology Information Basic Local Alignment Search Tool (NCBI BLAST v 2.18) program. The NCBI BLAST program is available on the internet from the National Center for Biotechnology Information (blast.ncbi.nlm.nih.gov/Blast.cgi). Typically the following parameters for NCBI BLAST can be used: Filter options set to “default”, the Comparison Matrix set to “BLOSUM62”, the Gap Costs set to “Existence: 11, Extension: 1”, the Word Size set to 3, the Expect (E threshold) set to 1e-3, and the minimum length of the local alignment set to 50% of the query sequence length.

The term “pesticidal”, as used herein, refers to the ability of a substance to decrease the rate of growth of a pest, i.e., an undersired organism, or to increase the mortality of a pest.

Promoter: As used herein, a “promoter” is a nucleotide sequence capable of initiating transcription in a cell and can drive or facilitate transcription of a nucleotide sequence or fragment thereof of the instant invention. Such promoters need not be of plant origin. For example, promoters derived from plant viruses, such as the CaMV35S promoter or from Agrobacterium tumefaciens, such as the T-DNA promoters, can be useful.

Polypeptide (also peptide, protein): The term “polypeptide”, as used herein, refers to a compound of two or more subunit amino acids, amino acid analogs, or other peptidomimetics, regardless of post-translational modification, e.g., phosphorylation or glycosylation. The subunits may be linked by peptide bonds or other bonds such as, for example, ester or ether bonds. Full-length polypeptides, truncated polypeptides, point mutants, insertion mutants, splice variants, chimeric proteins, and fragments thereof are encompassed by this definition.

Progeny: As used herein, “progeny” includes descendants of a particular plant or plant line. Progeny of an instant plant include seeds formed on F₁, F₂, F₃, F₄, F₅, F₆ and subsequent generation plants, or seeds formed on BC₁, BC₂, BC₃, and subsequent generation plants, or seeds formed on F₁BC₁, F₁BC₂, F₁BC₃, and subsequent generation plants. The designation F₁ refers to the progeny of a cross between two parents that are genetically distinct. The designations F₂, F₃, F₄, F₅ and F₆ refer to subsequent generations of self- or sib-pollinated progeny of an F₁ plant.

Regulatory region: The term “regulatory region”, as used herein, refers to a nucleotide sequence that influences transcription or translation initiation and rate, and stability and/or mobility of a transcription or translation product. Such regulatory regions need not be of plant origin. Regulatory sequences include but are not limited to promoter sequences, enhancer sequences, response elements, protein recognition sites, inducible elements, protein binding sequences, 5′ and 3′ untranslated regions (UTRs), transcriptional start sites, termination sequences, polyadenylation sequences, introns, and combinations thereof. A regulatory region typically comprises at least a core (basal) promoter. A regulatory region also may include at least one control element, such as an enhancer sequence, an upstream element or an upstream activation region (UAR). For example, a suitable enhancer is a cis-regulatory element (−212 to −154) from the upstream region of the octopine synthase (ocs) gene.

Stringency: Nucleic acid molecules or fragment thereof of the present invention are capable of specifically hybridizing to other nucleic acid molecules under certain circumstances. As used herein, two nucleic acid molecules are said to be capable of specifically hybridizing to one another if the two molecules are capable of forming an anti-parallel, double-stranded nucleic acid structure. A nucleic acid molecule is said to be the “complement” of another nucleic acid molecule if they exhibit complete complementarity. As used herein, molecules are said to exhibit “complete complementarity” when every nucleotide of one of the molecules is complementary to a nucleotide of the other. Two molecules are said to be “minimally complementary” if they can hybridize to one another with sufficient stability to permit them to remain annealed to one another under at least conventional “low-stringency” conditions. Similarly, the molecules are said to be “complementary” if they can hybridize to one another with sufficient stability to permit them to remain annealed to one another under conventional “high-stringency” conditions. Conventional stringency conditions are described by Sambrook et al., In: Molecular Cloning, A Laboratory Manual, 2nd Edition, Cold Spring Harbor Press, Cold Spring Harbor, N.Y. (1989), and by Haymes et al. In: Nucleic Acid Hybridization, A Practical Approach, IRL Press, Washington, D.C. (1985). Departures from complete complementarity are therefore permissible, as long as such departures do not completely preclude the capacity of the molecules to form a double-stranded structure. Thus, in order for a nucleic acid molecule or fragment of the present invention to serve as a primer or probe it needs only be sufficiently complementary in sequence to be able to form a stable double-stranded structure under the particular solvent and salt concentrations employed.

Appropriate stringency conditions which promote DNA hybridization include, for example, 6.0× sodium chloride/sodium citrate (SSC) at about 45° C., followed by a wash of 2.0×SSC at 50° C. These conditions are known to those skilled in the art, or can be found in Current Protocols in Molecular Biology, John Wiley & Sons, N.Y. (1989), 6.3.1-6.3.6. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or either the temperature or the salt concentration may be held constant while the other variable is changed.

Substantially purified: The term “substantially purified”, as used herein, refers to a molecule separated from substantially all other molecules normally associated with it in its native state. More preferably a substantially purified molecule is the predominant species present in a preparation. A substantially purified molecule may be greater than 60% free, preferably 75% free, more preferably 90% free, and most preferably 95% free from the other molecules (exclusive of solvent) present in the natural mixture. The term “substantially purified” is not intended to encompass molecules present in their native state.

Translational start site: As used herein, a “translational start site” is usually an ATG in the cDNA transcript, more usually the first ATG. A single cDNA, however, may have multiple translational start sites.

Transcription start site: As used herein, a “transcription start site” is the point at which transcription is initiated. This point is typically located about 25 nucleotides downstream from a TFIID binding site, such as a TATA box. Transcription can initiate at one or more sites within the gene, and a single gene may have multiple transcriptional start sites, some of which may be specific for transcription in a particular cell-type or tissue.

Transgenic organism: As used herein, a “transgenic organism” refers to an organism which comprises within its genome a heterologous polynucleotide. Generally, the heterologous polynucleotide is stably integrated within the genome such that the polynucleotide is passed on to successive generations. The heterologous polynucleotide may be integrated into the genome alone or as part of a recombinant expression cassette. “Transgenic” is used herein to include any cell, cell line, callus, tissue, the genotype of which has been altered by the presence of heterologous nucleic acid. The term transgenic includes those transgenics initially so altered as well as those created by sexual crosses or asexual propagation from the initial transgenic. The term transgenic as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutations.

Untranslated region (UTR): An “UTR”, as used herein is any contiguous series of nucleotide bases that is transcribed, but is not translated. These untranslated regions may be associated with particular functions such as increasing mRNA message stability. Examples of UTRs include but are not limited to polyadenylation signals, termination sequences, sequences located between the transcriptional start site and the first exon (5′ UTR), and sequences located between the last exon and the end of the mRNA (3′ UTR).

Volatile: “Volatile compounds” and “volatile organic compounds” (VOCs), as used herein, are compounds that in most instances evaporate readily at ambient temperature and pressure. Generally, volatile compounds are in the vicinity of the target pathogenic organism so long as they achieve their biological effect prior to evaporation. They may be spread on or around the base of the host plant or intermixed with the growth medium or soil of the plant. Physical contact with the host plant or target pathogen is not required due to the dispersal of the volatiles through the air or soil. Thus, volatile compounds produced by the culture of the present invention must be present “in the vicinity” of the target pathogenic organism for effectiveness. The organism producing the volatile compound is thus cultured in the vicinity of the host plant or the target organism, or in the growth medium or soil of the host plant prior to or concurrent with plant growth.

Reduced expression: As used herein, “reduced expression” refers to a decrease in production of expression products (mRNA, polypeptide, or both) relative to basal or native states.

Variant: A “variant”, as used herein, is a strain having identifying characteristics of the species to which it belongs, while having at least one nucleotide sequence variation or identifiably different trait with respect to the parental strain, where the trait is genetically based (heritable). For example, for a Muscodor strobelii strain, identifiable traits include 1) the ability to produce isobutyric acid, the ability to produce at least one derivative of isobutyric acid, such as but not limited to isobutyric acid methyl ester or isobutyric anhydride (allyl 2-methylpropanoate), or the ability to produce isobutyric acid and at least one derivative of isobutyric acid; or 2) the ability to kill certain species of microorganism such as, for example, one or more of Ganoderma boninense, Phytophthora palmivora, Pythium ultimum, and Rhizoctonia solani; or 3) having a genome with greater than 95%, greater than 96%, greater than 97%, greater than 98%, or greater than 99% sequence identity to the ribosomal RNA genes of M. strobelii can be used to confirm a variant as M. strobelii.

For nucleic acids and polypeptides, the term “variant” is used herein to denote a polypeptide, protein or polynucleotide molecule with some differences, generated synthetically or naturally, in their base or amino acid sequences as compared to a reference polypeptide or polynucleotide, respectively. For example, these differences include substitutions, insertions, deletions or any desired combinations of such changes in a reference polypeptide or polypeptide. Polypeptide and protein variants can further consist of changes in charge and/or post-translational modifications (such as glycosylation, methylation. phosphorylation, etc.).

All publications and patent applications mentioned in this specification are herein incorporated by reference to the same extent as if each individual publication or patent application was specifically and individually indicated to be incorporated by reference.

No admission is made that any reference constitutes prior art. The discussion of the references states what their authors assert, and the applicants reserve the right to challenge the accuracy and pertinence of the cited documents. It will be clearly understood that, although a number of prior art publications are referred to herein, this reference does not constitute an admission that any of these documents forms part of the common general knowledge in the art.

The discussion of the general methods given herein is intended for illustrative purposes only. Other alternative methods and embodiments will be apparent to those of skill in the art upon review of this disclosure.

Isolation and Characterization of Muscodor strobelii

Applicants have isolated and characterized a novel fungal species named Muscodor strobelii. Partial 18S rDNA and ITS sequences of M. strobelii (MB-8) were determined. Whole genome DNA of Muscodor strobelii was sequenced using shotgun approach. The structural and functional annotation of the assembled genome provided approximately 14,000 gene models, the sequences of which are provided in the attached Sequence Listing. A genome-wide expression analysis was performed on M. strobelii cells grown in conditions that induce the production of VOCs. Thus, this invention provides an isolated novel fungal species designated Muscodor strobelii, and mutants and variants thereof. The biologically pure or isolated culture can be in a variety of forms, including but not limited to, still cultures, whole cultures, stored stocks of mycelium and/or hyphae (particularly glycerol stocks), stored agar plugs in glycerol/water, freeze dried stocks, and dried stocks such as mycelia dried onto filter paper or grain seeds.

“Isolated” or grammatical equivalents as used herein and in the art is understood to mean that the referred to culture is a culture fluid, pellet, scraping, dried sample, lyophilate, or section (for example, hyphae or mycelia); or a support, container, or medium such as a plate, paper, filter, matrix, straw, pipette or pipette tip, fiber, needle, gel, swab, tube, vial, particle, etc. that contains a single type of organism. In the present invention, an isolated culture of M. strobelii is a culture fluid or a scraping, pellet, dried preparation, lyophilate, or section of M. strobelii, or a support, container, or medium that contains M. strobelii, in the absence of other organisms. In some embodiments, an M. strobelii strain is provided in which M. strobelii is not in contact with a plant host.

For example, as applied to the current invention, to obtain an isolated culture of M. strobelii tissue fragments from Philodendron sp. are placed in either culture fluid or agar (e.g., mycological agar) until fungal growth occurs, as is outlined in the examples. Fungal hyphae from the fungal growth are grown and serially transferred until a culture in pure form is obtained, as measured by observation (e.g., morphological and/or genetic unity). M. strobelii can be obtained, for example, as an endophyte from a plant species, such as a Philodendron plant in Malaysia. As provided in Example 1, the M. strobelii isolate designated MB-8 was isolated from small stems of Philodendron sp taken from a plant growing in the KL forest of the University of Malaysia.

The Muscodor strobelii strain may be characterized by the production of a whitish felt-like mycelium with intertwining hyphae on potato dextrose agar (PDA). Muscodor strobelii, as isolated in Example 1, did not develop fruiting structures or spores on water agar or any other media that have been tested. The Muscodor strobelii strain is considered to be related to the family of Xylariaceae based upon 99% similarity (563/565) of its partial 18S rDNA sequences to those of Muscodor albus, whose rDNA sequences show relatedness to the Xylariaceae. However, M. strobelii is distinguished from M. albus in that it totally lacks a stretch of intergenic sequence (364 bp) in its 18S rDNA which is partially diagnostic for Muscodor albus. Partial 18S rDNA and ITS sequences of M. strobelii (MB-8) were submitted to GenBank with the following accession numbers FJ664552 and FJ664551 respectively. These two sequences were found to be 100% and 99% identical to those of Muscodor yucatanensis, respectively. For GC/MS volatile fingerprinting, M. strobelii isolate MB-8 produces volatile compounds which are, for the most part, distinct from those produced by other Muscodor spp.

The M. strobelii fungal strain is described here for the first time as novel and distinct from M. albus, M. vitigenus, and M. yucatanensis isolates. In addition to diagnostic differences between M. strobelii, M. albus, and M. yucatanensis in its rDNA sequences, the novelty of this strain is also related to the production of substances, volatile organic compounds (VOCs) that are not only inhibitory but are lethal to Ganoderma boninense.

The invention provides a new species of fungus, M. strobelii that has the ability to kill Ganoderma boninense. No other isolates of Muscodor spp. produce VOCs that are lethal to Ganoderma boninense. In addition, a combination of molecular, biochemical and biological techniques was used to characterize it and the results show that this M. strobelii isolate has distinct and unusual characteristics. Further description of the M. strobelii strain is as follows.

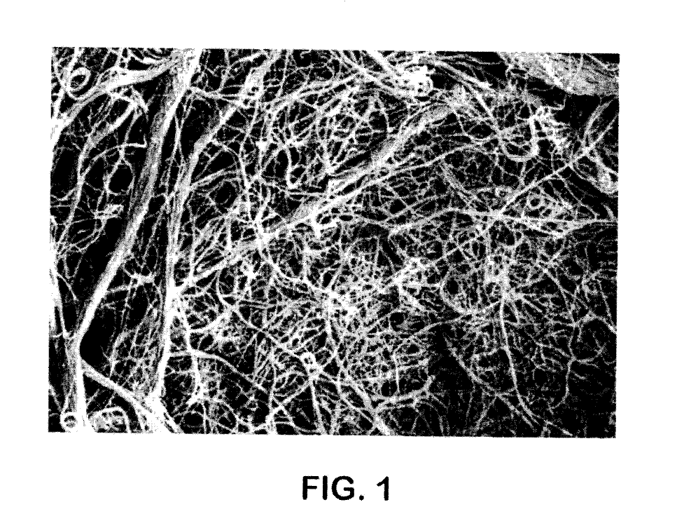

Fungus: Muscodor strobelii in nature is associated with a Philodendron sp. Spores: There are no spores or fruiting-bodies of this fungus observed under any culture conditions. Mycelium: It appears as a vast array of intertwined fine hyphae with coiled structures (see FIG. 1). The hyphae are 0.5-2.0 μm in diameter. Genetically: It is related to other Muscodor spp. (Xylariaceae). It differs from other Muscodor spp. by producing a distinct blend of volatile organic substances and its rDNA sequence possesses uncommon structural features.

Holotype: Endophytic on Philodendron sp. Collections were made at the University Forest of the University of Malaysia. The holotype comes from only one Philodendron sp. stem, collected in the University of Malaysia Forest south of Kuala Lumpur by Dr. Gary Strobel and Dr. Nora Zin. A living culture was deposited as Muscodor strobelii MB-8 on Jun. 5, 2009 in the Agricultural Research Service Culture Collection located at 1815 N. University Street, Peoria, IL 61604, USA (NRRL) in accordance with the Budapest Treaty on the International Recognition of the Deposit of Microorganisms for the Purpose of patent Procedure and the Regulations thereunder (Budapest Treaty) as Accession Number NRRL 50288.

The strain has been deposited under conditions to ensure that access to the culture will be available during the pendency of this patent application to one determined by the Commissioner of Patents and Trademarks to be entitled thereto under 37 C.F.R. §1.14 and 35 U.S.C. §122. The deposit represents a substantially pure culture of the deposited strain. The deposit is available as required by foreign patent laws in countries wherein counterparts of the subject application or its progeny are filed. However, it should be understood that the availability of a deposit does not constitute a license to practice the subject invention in derogation of patent rights granted by governmental action. Based on this deposit, the entire genome of the isolate NRRL 50288 is hereby incorporated into and included herein.

Teleomorph: The teleomorph of this fungus may be found in Xylariaceae, based on the similarity of the 18S rDNA gene sequence data between M. albus and the family Xylariaceae in the GenBank database (Bruns et al., 1991, Ann. Rev. Ecology Systematics 22: 525-564; Reynolds and Taylor, 1993, Proceedings of an International Symposium Wallingford: C.A.B. International; Mitchell et al, 1995, Mycologist 9: 67-75; Guano et al., 1999, Clin. Microbiol. Rev. 12(3): 454-500; Taylor et al., 1999, Ann. Rev. Phytopathology 37: 197-246). The molecular data from the 18S rDNA gene sequences of M. strobelii show a 99% homology with M. albus isolate 620, but M. strobelii lacks an intergenic sequence (18S rDNA) that is diagnostic for M. albus. A similar sequence is also absent in the partial 18S rDNA of M. vitigenus. Partial 18S rDNA and ITS sequences of M. strobelii (MB-8) were found to be 100% and 99% identical to those of Muscodor yucatanensis, respectively.

Etymology: The genus name, Muscodor, is taken from the Latin word which means musty. This is consistent with the quality of the odor produced by the first three isolates of the genus. The species name is strobelii, given in honor of Gary Strobel.

Molecular Biology of Muscodor strobelii

The partial sequences of 18S rDNA, ITS1, 5.8S, and ITS2 have been demonstrated to be highly conserved regions of DNA and therefore very useful in the classification of organisms (Mitchell et al., 1995, Mycologist 9: 67-75). These molecularly distinguishing partial sequences of M. strobelii were obtained and compared with the data in GenBank. After searching the 18S rDNA sequences, 565 bp of M. strobelii were subjected to an advanced BLAST search. The results showed 100% and 99% identity with those of M. yucatanensis (FJ917287.1) and M. albus (AF324337), respectively. However, M. strobelii lacks an intergenic sequence in the 18S rDNA (364 bp) that is diagnostic for the original Muscodor albus 620. In addition, a BLAST search of the 597 bp sequence of M. strobelii corresponding to SEQ ID NO:40284 revealed that it showed 99% identity with each of the following GenBank sequences FJ917287.1, EU687035.1, EU686810.1, and EU686807.1. The sequence of SEQ ID NO:40284 also showed 97% identity with the corresponding region of M. vitigenus. Thus, the M. strobelii ITS sequence like the 18S rDNA sequence more closely resembles M. yucatanesis and M. vitigenus than M albus.

Thus, M. strobelii isolated from other organisms can be identified as a Muscodor species, using well known techniques, including classification with the Muscodor genus on the basis of the relatedness of the 18S rDNA sequence and ITS region to previously identified members of the Muscodor genus, as well as similarity of its hyphae to other Muscodor species. For example, an M. strobelii isolate can be identified as Muscodor strobelii by determining regions of the 18S rDNA sequence, including the region that includes ITS sequence provided as SEQ ID NO:40284, and the region that includes the 18S rDNA partial sequence provided as SEQ ID NO:40285 in the Sequence Listing.

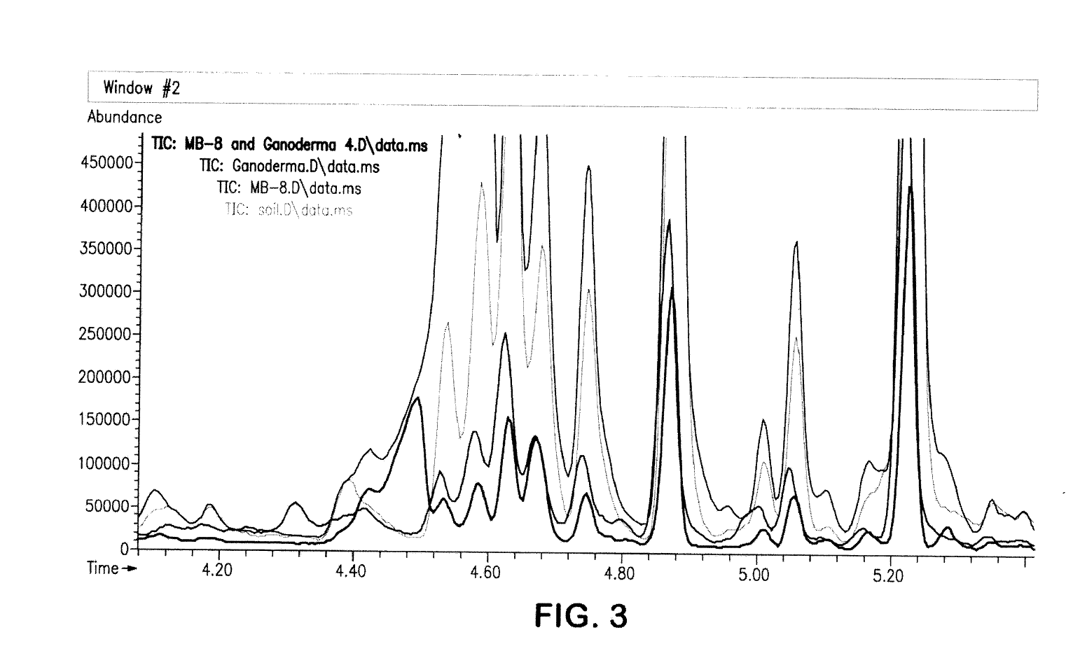

An M. strobelii isolate, such as the isolate of Examples 1-12, can also be identified by one or more VOCs produced by the isolate. The volatile organic compounds produced by a fourteen day old culture of M. strobelii is provided in Table 1 of Example 4 herein, and a gas chromatogram of the volatile compounds removed from a M. strobelii culture container is shown in FIG. 3. For example, a culture of M. strobelii can be identified by determining that the fungal isolate is a Muscodor species by morphological characteristics and/or molecular genetic analysis, as illustrated in Examples 1-3, 5, and 6, and/or by its production of the VOC isobutyric acid and/or one or more derivatives of isobutyric acid (including, without limitation, isobutyric anhydride (allyl 2-methylpropanoate), isobutyric acid, methyl ester (methylisobutyrate); isobutyric acid, ethyl ester; isobutyric acid, propyl ester; and isobutyric acid, allyl ester), as demonstrated in Example 4, for example. In some embodiments, a Muscodor strain, isolate, or variant can be identified as M. strobelii by its ability to produce, or by its production of, isobutyric acid. In some examples, a Muscodor strain, isolate, or variant can be identified as M. strobelii by determining that the most abundant component of the VOC s produced by the organism is isobutyric acid or a derivative thereof. For example, a M. strobelii strain, isolate, or variant can be identified as a Muscodor species in which the most abundant component of the VOCs produced by the organism is isobutyric acid.

In some embodiments, an M. strobelii strain, isolate, or variant is identified as a Muscodor species that produces VOC s in which at least 30 mole percent, at least 35 mole percent, at least 40 mole percent, at least 45 mole percent, at least 50 mole percent, or at least 55 mole percent of the volatile organic compounds produced by the strain, isolate, or variant is isobutyric acid. In some embodiments, an M. strobelii strain, isolate, or variant is identified as producing VOC s in which at least 30 mole percent, at least 35 mole percent, at least 40 mole percent, at least 45 mole percent, at least 50 mole percent, at least 55 mole percent, at least 60 mole percent, at least 65 mole percent, at least 70 mole percent of the VOC s are isobutyric acid and isobutyric acid derivatives, such as but not limited to isobutyric acid, methyl ester; isobutyric acid, ethyl ester; isobutyric acid, propyl ester; and isobutyric acid, allyl ester. For example, a M. strobelii strain, isolate, or variant is in some embodiments identified as producing VOCs in which at least 30 mole percent, at least 35 mole percent, at least 40 mole percent, at least 45 mole percent, at least 50 mole percent, at least 55 mole percent, at least 60 mole percent, at least 65 mole percent, at least 70 mole percent of the VOC s are isobutyric acid and isobutyric acid, methyl ester.

Cultures of M. strobelii may in some instances also be characterized by production of sesquiterpenes. A sesquiterpene used to identify M. strobelii is in some embodiments a pheromone, such as a pheromone that affects the behavior of one or more insect species. For example, sesquiterpene compounds such as isocaryophyllene or derivatives thereof (e.g., (−)-tricyclo[6.2.1.0(4,11)]undec-5-ene,1,5,9,9-tetramethyl-(isocaryophyllene-II), bergamotene or derivatives thereof, patchoulene or derivatives thereof (e.g., alpha-patchoulene), gurjunene or derivatives thereof (e.g., alpha-gurjunene), aristolene or derivatives thereof (e.g., (−)-aristolene), or isolongifolene or derivatives thereof (e.g., 4,5-dehydro-isolongifolene) that may be present in the mixture of VOCs produced by a Muscodor strain can be used to identify the species as M. strobelii. Detection of one or more of bergamotene, patchoulene, gurjunene, aristolene, or isolongifolene in the VOCs produced by a Muscodor isolate can optionally be used in the identification of M. strobelii.

The biological activity of the volatile organic compounds produced by a Muscodor isolate can also be used to identify an isolate as Muscodor strobelii. For example, in the experiments detailed in Example 7, several Petri plates of PDA were used to determine if the fungus produced volatile antibiotics that were able to inhibit the growth of other organisms. This procedure included removing a 1-inch section of the agar from the middle of the plate, plating a plug of the M. strobelii isolate MB-8 isolate on one side and allowing it to grow for several days, and then plating test organisms on the other side of the gap (Strobel et al., 2001, Microbiology 147: 2943-2950). The M. strobelii isolate MB-8 demonstrated the ability to produce volatile antibiotics, which either inhibited or killed the fungi that were placed on the other side of the center well as test organisms, such as Pythium ultimum, Sclerotinia sclerotiorum, and others. In these assays as well as in experiments using oil palm (Elaeis guineensis) roots exposed to Ganoderma boninense and experiments using soil treated with Ganoderma boninense, both also described in Example 7, it has been demonstrated that M. strobelii has the ability to kill Ganoderma boninense and to prevent the growth of Ganoderma boninense on palm tissue and in the soil. Thus, in some embodiments, a Muscodor isolate that has the ability to kill Ganoderma boninense is identified as M. strobelii.

Method of Isolating Muscodor strobelii

In another aspect, the invention provides a method of isolating Muscodor strobelii. The method comprises culturing tissue from a portion of a plant that is a host to Muscodor strobelii, such as a Philodendron, Eucryphia, or Dacrydium species. For example, tissue can be removed from the interior region of a Philodendron sp. plant stem and cultured on nutrient media for a time sufficient to permit colony formation by a strain of Muscodor strobelii associated with the tissue. The Muscodor strobelii strain is then selected. Selection of the strain may be accomplished by one of skill in the art according to the above description of characteristics of the M. strobelii MB-8 isolate.

For example, selection of Muscodor strobelii may be undertaken by plating the strain in culture with a test organism. Test organisms known to have growth inhibited by, or are killed by, Muscodor strobelii are particularly desirable. Two such exemplary test organisms include Pythium ultimum and Sclerotinia sclerotiorum.

Confirmation of successful isolation of Muscodor strobelii may also be undertaken by measuring the volatile organic compounds released by the isolate. As measured by GC/MS, the fungus consistently produces primarily isobutyric acid and one or more isobutyric acid derivatives along with several unusual lipids including aristolene, beta-patchoulene and others (see Table 1). Both of the latter compounds are described as insect pheromones. These compounds are not produced by any of the described isolates of M. albus (Strobel et al., 2001, Microbiology 147: 2943-2950; Ezra et al., 2004, Microbiology 150:4023-4031) or M. vitigenus (U.S. Pat. No. 7,267,975). Likewise, none of other naphthalene and azulene derivatives of M. albus were produced by M. strobelii when grown on PDA. Unlike M. vitigenus, M. strobelii does not produce a mixture of VOCs in which naphthalene is the predominant VOC. Thus, chemically this organism is different than other Muscodor spp. that have been studied and described thus far. The odor produced by the fungus becomes noticeable after about 3-4 days and seems to increase with time up to and including at least three weeks. The volatile compounds of this fungus possess inhibitory and lethal bioactivity against a number of plant and human pathogens using the standard bioassay technique (Strobel et al., 2001, Microbiology 147: 2943-2950).

Muscodor strobelii Compositions

The invention compositions comprising the culture of M. strobelii can be in a variety of forms, including, but not limited to, still cultures, whole cultures, stored stocks of mycelium and/or hyphae (particularly glycerol stocks), agar strips, stored agar plugs in glycerol/water, freeze dried stocks, and dried stocks such as mycelia dried onto filter paper or grain seeds. The disclosure further provides a composition that includes Muscodor strobelii and a carrier. The carrier may be any one or more of a number of carriers that confer a variety of properties, such as increased stability, wettability, dispersability, etc. Wetting agents such as natural or synthetic surfactants, which can be nonionic or ionic surfactants, or a combination thereof can be included in a composition of the invention. Water-in-oil emulsions can also be used to formulate a composition that includes M. strobelii (see, for example, U.S. patent No. http://patft.uspto.gov/netacgi/nph-Parser?Sect1=PTO1&Sect2=HITOFF&d=PALL&p=1&u=%2Fnetahtml%2FPTO%2Fsrchnum. htm&r=1&f=G&1=50&s1=7485451.PN.&OS=PN/7485451 &RS=PN/7485451-h0#h0http://patft.uspto.gov/netacgi/nph-Parser?Sect1=PTO1&Sect2=HITOFF&d=PALL&p=1&u=%2Fnetahtml%2FPTO%2Fsrehnum. htm&r=1&f=G&1=50&s1=7485451.PN.&OS=PN/7485451&RS=PN/7485451-h2#h27,485,451, incorporated by reference herein). Suitable formulations that may be prepared include wettable powders, granules, gels, agar strips or pellets, thickeners, and the like, microencapsulated particles, and the like, liquids such as aqueous flowables, aqueous suspensions, water-in-oil emulsions, etc. The formulation may include grain or legume products (e.g., ground grain or beans, broth or flour derived from grain or beans), starch, sugar, or oil. The carrier may be an agricultural carrier. In certain embodiments the carrier is a seed, and the composition may be applied or coated onto the seed or allowed to saturate the seed.

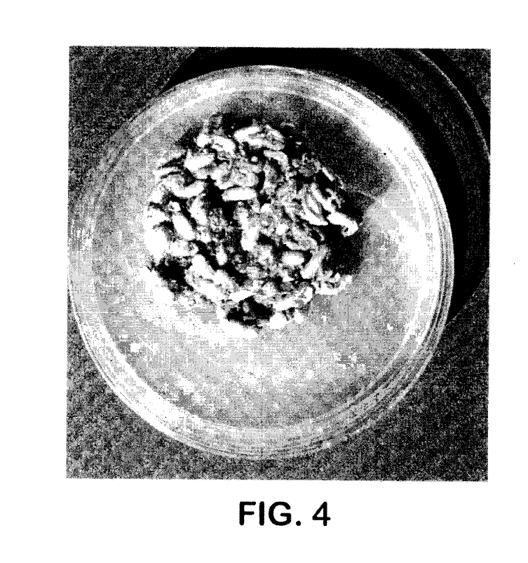

The agricultural carrier may be soil or plant growth medium. Other agricultural carriers that may be used include water, fertilizers, plant-based oils, humectants, or combinations thereof. Alternatively, the agricultural carrier may be a solid, such as diatomaceous earth, loam, silica, alginate, clay, bentonite, vermiculite, seed cases, other plant and animal products, or combinations, including granules, pellets, or suspensions. Mixtures of any of the aforementioned ingredients are also contemplated as carriers, such as but not limited to, pesta (flour and kaolin clay), agar or flour-based pellets in loam, sand, or clay, etc. Formulations may include food sources for the cultured organisms, such as barley, rice, or other biological materials such as seed, plant parts, empty fruit bunches (EFB) from palm, sugar cane bagasse, hulls or stalks from grain processing, ground plant material (“yard waste”) or wood from building site refuse, sawdust or small fibers from recycling of paper, fabric, or wood. The agricultural carrier may be a mixture of EFB and rice, as shown in FIG. 4. Other suitable formulations will be known to those skilled in the art.

When used as pesticides or fungicide in their commercially available formulations and in the use forms prepared with these formulations, the active compounds and compositions according to the invention can furthermore be present in the form of a mixture with synergists. Synergists are compounds by which the activity of the active compounds is increased without it being necessary for the synergist added to be active itself.

When used as pesticides in their commercially available formulations and in the use forms prepared with these formulations, the active compounds and compositions according to the invention can furthermore be present in the form of a mixture with inhibitors which reduce the degradation of the active compound after application in the habitat of the plant, on the surface of parts of plants or in plant tissues.

The active compounds and compositions according to the invention, as such or in their formulations, can also be used as a mixture with known acaricides, bactericides, fungicides, insecticides, microbicides, or nematicides, or combinations thereof, for example in order to widen the spectrum of action or to prevent the development of resistances in this way. In many cases, synergistic effects result, i.e. the activity of the mixture can exceed the activity of the individual components. A mixture with other known active compounds, such as fertilizers, growth regulators, safeners and/or semiochemicals is also possible.

In a preferred embodiment of the present invention, the composition may further include at least one chemical or biological pesticide. A variety of pesticides is apparent to one of skill in the art and may be used. Exemplary chemical pesticides include those in the organophosphate, carbamate, organochlorine, and prethroid classes. Also included are chemical control agents such as, but not limited to, benomyl, borax, captafol, captan, chorothalonil, formulations containing copper; formulations containing zinc; dichlone; dicloran; iodine; fungicides that inhibit ergosterol biosynthesis such as but not limited to fenarimol, imazalil, myclobutanil, propiconazole, prochloraz, terbutrazole, flusilazole, triadimefon, and tebuconazole; folpet; ipordione; manocozeb; maneb; metalaxyl; oxycarboxin, oxytetracycline; PCNB; pentachlorophenol; quinomethionate; sodium aresenite; sodium DNOC; sodium hypochlorite; sodium phenylphenate; streptomycin; sulfur; thiabendazolel; thiophanate-methyl; triforine; vinclozolin; zineb; ziram; tricyclazole; cymoxanil; blastididin; and validimycin.

Exemplary biological pesticides that can be included in a M. strobelii composition of the invention for preventing a plant pathogenic disease include microbes, animals, plants, bacteria, genetic material, and natural products of living organisms. In these compositions, the M. strobelii is isolated prior to formulation with an additional organism. For example, microbes such as but not limited to species of Bacillus, Trichoderma, Erwinia, Pichia, Candida, Cryptococcus, Cordyceps, Talaromyces, Paecilomyces, Beauveria, Chaetomium, Gliocladium, Aureobasidium, Dabaryomyces, Exophilia, Ampelomyces, and Mariannaea can be provided in a composition with Muscodor strobelii.

Examples of fungi that can be combined with Muscodor strobelii in a composition include, without limitation, other Muscodor species, Fusarium lateritium, Metarhizium anisopliae (“green muscarine”), Metarhizium flaviride, Beauveria bassiana (“white muscarine”), Beauveria brongniartii, Chladosporium herbarum, Paecilomyces farinosus, Paecilomyces fumosoroseus, Verticillium lecanii, Hirsutella citriformis, Hirsutella thompsoni, Aschersonia aleyrodis, Entomophaga grylli, Entomophaga maimaiga, Entomophaga muscae, Entomophaga praxibulli, Entomophthora plutellae, Zoophthora radicans, Neozygitesfloridana, Nomuraea rileyi, Pandora neoaphidis, Tolypocladium cylindrosporum, Culicinomyces clavosporus, Muscodor albus, Cordyceps variabilis, Cordyceps facis, Cordyceps subsessilis, Cordyceps myrmecophila, Cordyceps sphecocephala, Cordyceps entomorrhiza, Cordyceps gracilis, Cordyceps militaris, Cordyceps washingtonensis, Cordyceps melolanthae, Cordyceps ravenelii, Cordyceps unilateralis, Cordyceps sinensis and Cordyceps clavulata, and mycorrhizal species such as Laccaria bicolor. Other mycopesticidal species will be apparent to those skilled in the art.

Exemplary food preservatives include antimicrobial preservatives, which inhibit the growth of bacteria and fungi and mold growth, or antioxidants such as oxygen absorbers, which inhibit the oxidation of food constituents. Common antimicrobial preservatives include calcium propionate, sodium nitrate, sodium nitrite, sulfites (sulfur dioxide, sodium bisulfite, potassium hydrogen sulfite, etc.) and disodium EDTA. Antioxidants include BHA and BHT. Other preservatives include formaldehyde (usually in solution), glutaraldehyde (kills insects), ethanol and methylchloroisothiazolinone.

Treating Plant Pathogens with Muscodor strobelii

Muscodor strobelii may release volatile organic compounds that control major diseases of plants. One exemplary plant is the oil palm, whereby compounds released by M. strobelii may inhibit the development of basal stem rot disease. These compounds may target Ganoderma boninense, a causative agent of basal stem rot disease. (See Examples 7 and 8) M. strobelii may be significantly more efficacious in targeting basal stem rot disease than other members of the Muscodor genus, such as M. albus.

Generally, volatile compounds are in the vicinity of the target pathogenic organism so long as they achieve their biological effect prior to evaporation. The organism may be spread on or around the base of the host plant or intermixed with the growth medium or soil of the plant. Physical contact of the organism with the host plant or target pathogen is not required due to the dispersal of the volatiles through the air or soil.

In order to achieve good dispersion and adhesion of compositions within the present invention, it may be advantageous to formulate the culture and/or volatile compound with components that aid dispersion and adhesion. Suitable formulations are apparent to those of skill in the art and include wettable powders, granules and the like, microencapsulations in a suitable medium and the like, liquids such as aqueous flowables and aqueous suspensions, volatile compositions, and emulsifiable concentrates. Formulations may include food sources for the cultured organisms, such as barley, rice, or other organic materials such as empty fruit bunches. Other suitable formulations will be known to those skilled in the art.

In one aspect the invention provides methods for preventing or treating a plant-pathogen-related disease, in which the method includes applying a composition comprising one or more Muscodor strobelii organisms to a plant. The composition may comprise one or more of carriers, Muscodor strobelii viability maintenance compounds, chemical pesticides, and/or one or more additional biological pesticides, as disclosed herein. In some embodiments, the composition is an aqueous suspension of M. strobelii. In order to achieve good dispersion and adhesion of compositions within the present invention, it may be advantageous to formulate the culture with components that aid dispersion and adhesion. As detailed above, suitable formulations are apparent to those of skill in the art and include wettable powders, granules and the like, microencapsulations in a suitable medium and the like, liquids such as aqueous flowables and aqueous suspensions and emulsifiable concentrates. Formulations may include food sources for the cultured organisms, such as barley, rice, flour, sugar, or other organic materials.

Viability maintenance compounds may serve to stabilize Muscodor strobelii during shipping, transport, storage, and treatment of the plants or crops. For example, Muscodor strobelii may be placed in culture medium and glycerol for storage at −70° C. or in culture medium and distilled water for storage at 0° C.

In some embodiments, the composition is a liquid composition, such as a suspension or emulsion, and the composition is used to water, spray, or drench the plant. The M. strobelii composition may be provided to the user as a solid or semi-solid formulation to which water or a liquid solution is added by the user prior to application. In other embodiments, the composition is a powder or particulate composition, and the composition is dusted or scattered on the plant, on the soil surface, or at the base of the plant. In some embodiments, the M. strobelii composition is then watered into the soil.

M. strobelii may be topically administered to plants, including any or all plant parts, including without limitation, roots, shoots, stems, bark, leaves, fruit, and/or seeds, in either dry or solution form. For example, M. strobelii may be applied on, in, or near the root of the oil palm. An M. strobelii composition can be applied to plants through any means, including watering, drenching, spraying, or dusting. Single or multiple applications are contemplated. Multiple applications can be through more than one application method. The treatment or inoculation of plants with M. strobelii may allow the systemic growth of the fungus as a symbiotic or endophytic organism throughout the plant. In this case, the fungus may establish itself within the plant as a harmless endophyte and preclude attack of the plant by otherwise harmful bacteria, fungi, or insects. In some embodiments, a plant may be abraded, pierced, or otherwise wounded during or prior to application of M. strobelii to promote establishment of M. strobelii as an endophyte on the plant.

A seed coating or seed dressing formulation can be applied to the seeds employing the compositions of the invention and a diluent in suitable seed coating formulation form, e.g. as an aqueous suspension or in a dry powder form having good adherence to the seeds. Such seed coating or seed dressing formulations are known in the art. Such formulations may contain the single active ingredients or the combination of active ingredients in encapsulated form, e.g. as slow release capsules or microcapsules.

The compositions of the invention are particularly useful in combating plant pests and plant pathogens, particularly phytopathogenic fungi. Thus, the invention has may be used to treat, inhibit or prevent the development of plant pathogenic diseases caused by a broad range of fungi. The compositions and methods of the present invention are preferably used against fungi that are important or interesting for agriculture, horticulture, plant biomass for the production of biofuel molecules and other chemicals, and/or forestry. Non-limiting examples include, for instance, Acremonium strictum, Agrobacterium tumefaciens, Alternaria alternata, Alternaria solani, Aphanomyces euteiches, Aspergillus fumigatus, Athelia rolfsii, Aureobasidium pullulans, Bipolaris zeicola, Botrytis cinerea, Calonectria kyotensis, Cephalosporium maydis, Cercospora medicaginis, Cercospora sojina, Colletotrichum coccodes, Colletotrichum fragariae, Colletotrichum graminicola, Coniella diplodiella, Coprinopsis psychromorbida, Corynespora cassiicola, Curvularia pallescens, Cylindrocladium crotalariae, Diplocarpon earlianum, Diplodia gossyina, Diplodia spp., Epicoccum nigrum, Erysiphe cichoracearum, Fusarium graminearum, Fusarium oxysporum, Fusarium oxysporum f. sp. tuberosi, Fusarium proliferatum var. proliferatum, Fusarium solani, Fusarium verticillioides, Ganoderma boninense, Geotrichum candidum, Glomerella tucumanensis, Guignardia bidwellii, Kabatiella zeae, Leptosphaerulina briosiana, Leptotrochila medicaginis, Macrophomina, Macrophomina phaseolina, Magnaporthe grisea, Magnaporthe oryzae, Microsphaera manshurica, Monilinia fructicola, Mycosphaerella fijiensis, Mycosphaerella fragariae, Nigrospora oryzae, Ophiostoma ulmi, Pectobacterium carotovorum, Pellicularia sasakii (Rhizoctonia solani), Peronospora manshurica, Phakopsora pachyrhizi, Phoma foveata, Phoma medicaginis, Phomopsis longicolla, Phytophthora cinnamomi, Phytophthora erythroseptica, Phytophthora fragariae, Phytophthora infestans, Phytophthora medicaginis, Phytophthora megasperma, Phytophthora palmivora, Podosphaera leucotricha, Pseudopeziza medicaginis, Puccinia graminis subsp. Tritici (UG99), Puccinia sorghi, Pyricularia grisea, Pyricularia oryzae, Pythium ultimum, Rhizoctonia solani, Rhizoctonia zeae, Rosellinia sp., Sclerotinia sclerotiorum, Sclerotinina trifoliorum, Sclerotium rolfsii, Septoria glycines, Septoria lycopersici, Setomelanomma turcica, Sphaerotheca macularis, Spongospora subterranea, Stemphylium sp, Synchytrium endobioticum, Thecaphora (Angiosorus), Thielaviopsis, Tilletia indica, Trichoderma viride, Ustilago maydis, Verticillium albo-atrum, Verticillium dahliae, Verticillium dahliae, Xanthomonas axonopodis, Xanthomonas oryzae pv. oryzae.

In a preferred embodiment of the present invention, the application of the M. strobelii organisms to the plant results in a reduced occurrence of at least one plant disease caused by a bacterium, fungus, or insect as compared with plants not treated with an M. strobelii composition. In some embodiments, the plant disease is caused by Aspergillus fumigatus, Botrytis cinerea, Cerpospora betae, Curvularia spp., Ganoderma boninense, Geotrichum candidum, Mycosphaerella fijiensis, Phytophthora palmivora, Phytophthora ramorum, Pythium ultimum, Rhizoctonia solani, Rhizopus spp., Schizophyllum spp., Sclerotinia sclerotiorum, Verticillium dahliae, or Xanthomonas axonopodis. In a particularly preferred embodiment, the host plant is susceptible to diseases caused by Ganoderma boninense. In another preferred embodiment, the host plant is an oil palm plant. In other preferred embodiments, the disclosed compositions and methods are effective to kill the plant pathogen.

It is understood that all plants and plant parts can be treated in accordance with the invention. Plants are to be understood as meaning in the present context all plants and plant populations such as desired and undesired wild plants or crop plants (including naturally occurring crop plants). Crop plants can be plants which can be obtained by conventional plant breeding and optimization methods or by biotechnological and recombinant methods or by combinations of these methods, including the transgenic plants and plant cultivars protectable or not protectable by plant breeders' rights. Plant parts are to be understood as meaning all parts and organs of plants above and below the ground, such as shoot, leaf, flower and root, examples which may be mentioned being leaves, needles, stalks, stems, flowers, fruit bodies, fruits, seeds, roots, tubers and rhizomes. The plant parts also include harvested material, and vegetative and generative propagation material, for example cuttings, tubers, rhizomes, offsets and seeds.

As discussed above, the compositions and methods according to the present invention in principle can be applied to any plant. Therefore, monocotyledonous as well as dicotyledonous plant species are particularly suitable. The process is preferably used with plants that are important or interesting for agriculture, horticulture, for the production of biomass used in producing liquid fuel molecules and other chemicals, and/or forestry.

Thus, the invention has use over a broad range of plants, preferably higher plants pertaining to the classes of Angiospermae and Gymnospermae. Plants of the subclasses of the Dicotylodenae and the Monocotyledonae are particularly suitable. Dicotyledonous plants belong to the orders of the Magniolales, Illiciales, Laurales, Piperales Aristochiales, Nymphaeales, Ranunculales, Papeverales, Sarraceniaceae, Trochodendrales, Hamamelidales, Eucomiales, Leitneriales, Myricales, Fagales, Casuarinales, Caryophyllales, Batales, Polygonales, Plumbaginales, Dilleniales, Theales, Malvales, Urticales, Lecythidales, Violales, Salicales, Capparales, Ericales, Diapensales, Ebenales, Primulales, Rosales, Fabales, Podostemales, Haloragales, Myrtales, Cornales, Proteales, Santales, Rafflesiales, Celastrales, Euphorbiales, Rhamnales, Sapindales, Juglandales, Geraniales, Polygalales, Umbellales, Gentianales, Polemoniales, Lamiales, Plantaginales, Scrophulariales, Campanulales, Rubiales, Dipsacales, and Asterales. Monocotyledonous plants belong to the orders of the Alismatales, Hydrocharitales, Najadales, Triuridales, Commelinales, Eriocaulales, Restionales, Poales, Juncales, Cyperales, Typhales, Bromeliales, Zingiberales, Arecales, Cyclanthales, Pandanales, Arales, Lilliales, and Orchidales. Plants belonging to the class of the Gymnospermae are Pinales, Ginkgoales, Cycadales and Gnetales.

Suitable species may include members of the genus Abelmoschus, Abies, Acer, Agrostis, Allium, Alstroemeria, Ananas, Andrographis, Andropogon, Artemisia, Arundo, Atropa, Berberis, Beta, Bixa, Brassica, Calendula, Camellia, Camptotheca, Cannabis, Capsicum, Carthamus, Catharanthus, Cephalotaxus, Chrysanthemum, Cinchona, Citrullus, Coffea, Colchicum, Coleus, Cucumis, Cucurbita, Cynodon, Datura, Dianthus, Digitalis, Dioscorea, Elaeis, Ephedra, Erianthus, Erythroxylum, Eucalyptus, Festuca, Fragaria, Galanthus, Glycine, Gossypium, Helianthus, Hevea, Hordeum, Hyoscyamus, Jatropha, Lactuca, Linum, Lolium, Lupinus, Lycopersicon, Lycopodium, Manihot, Medicago, Mentha, Miscanthus, Musa, Nicotiana, Oryza, Panicum, Papaver, Parthenium, Pennisetum, Petunia, Phalaris, Phleum, Pinus, Poa, Poinsettia, Populus, Rauwolfia, Ricinus, Rosa, Saccharum, Salix, Sanguinaria, Scopolia, Secale, Solanum, Sorghum, Spartina, Spinacea, Tanacetum, Taxus, Theobroma, Triticosecale, Triticum, Uniola, Veratrum, Vinca, Vitis, and Zea.

The methods of the present invention are preferably used in plants that are important or interesting for agriculture, horticulture, biomass for the production of biofuel molecules and other chemicals, and/or forestry. Non-limiting examples include, for instance, Panicum virgatum (switchgrass), Sorghum bicolor (sorghum, sudangrass), Miscanthus giganteus (miscanthus), Saccharum sp. (energycane), Populus balsamifera (poplar), Zea mays (corn), Glycine max (soybean), Brassica napus (canola), Triticum aestivum (wheat), Gossypium hirsutum (cotton), Oryza sativa (rice), Helianthus annuus (sunflower), Medicago sativa (alfalfa), Beta vulgaris (sugarbeet), Pennisetum glaucum (pearl millet), Panicum spp., Sorghum spp., Miscanthus spp., Saccharum spp., Erianthus spp., Populus spp., Andropogon gerardii (big bluestem), Pennisetum purpureum (elephant grass), Phalaris arundinacea (reed canarygrass), Cynodon dactylon (bermudagrass), Festuca arundinacea (tall fescue), Spartina pectinata (prairie cord-grass), Arundo donax (giant reed), Secale cereale (rye), Salix spp. (willow), Eucalyptus spp. (eucalyptus), Triticosecale spp. (triticum—wheat X rye), Bamboo, Carthamus tinctorius (safflower), Jatropha curcas (Jatropha), Ricinus communis (castor), Elaeis guineensis (oil palm), Phoenix dactylifera (date palm), Archontophoenix cunninghamiana (king palm), Syagrus romanzoffiana (queen palm), Linum usitatissimum (flax), Brassica juncea, Manihot esculenta (cassaya), Lycopersicon esculentum (tomato), Lactuca saliva (lettuce), Musa paradisiaca (banana), Solanum tuberosum (potato), Brassica oleracea (broccoli, cauliflower, brusselsprouts), Camellia sinensis (tea), Fragaria ananassa (strawberry), Theobroma cacao (cocoa), Coffea arabica (coffee), Vitis vinifera (grape), Ananas comosus (pineapple), Capsicum annum (hot & sweet pepper), Allium cepa (onion), Cucumis melo (melon), Cucumis sativus (cucumber), Cucurbita maxima (squash), Cucurbita moschata (squash), Spinacea oleracea (spinach), Citrullus lanatus (watermelon), Abelmoschus esculentus (okra), Solanum melongena (eggplant), Papaver somniferum (opium poppy), Papaver orientale, Taxus baccata, Taxus brevifolia, Artemisia annua, Cannabis saliva, Camptotheca acuminate, Catharanthus roseus, Vinca rosea, Cinchona officinalis, Coichicum autumnale, Veratrum californica, Digitalis lanata, Digitalis purpurea, Dioscorea spp., Andrographis paniculata, Atropa belladonna, Datura stomonium, Berberis spp., Cephalotaxus spp., Ephedra sinica, Ephedra spp., Erythroxylum coca, Galanthus wornorii, Scopolia spp., Lycopodium serratum (Huperzia serrata), Lycopodium spp., Rauwolfia serpentina, Rauwolfia spp., Sanguinaria canadensis, Hyoscyamus spp., Calendula officinalis, Chrysanthemum parthenium, Coleus forskohlii, Tanacetum parthenium, Parthenium argentatum (guayule), Hevea spp. (rubber), Mentha spicata (mint), Mentha piperita (mint), Bixa orellana, Alstroemeria spp., Rosa spp. (rose), Dianthus caryophyllus (carnation), Petunia spp. (petunia), Poinsettia pulcherrima (poinsettia), Nicotiana tabacum (tobacco), Lupinus albus (lupin), Uniola paniculata (oats), bentgrass (Agrostis spp.), Populus tremuloides (aspen), Pinus spp. (pine), Abies spp. (fir), Acer spp. (maple), Hordeum vulgare (barley), Poa pratensis (bluegrass), Lolium spp. (ryegrass), Phleum pratense (timothy), and conifers. Of interest are plants grown for energy production, so called energy crops, such as cellulose-based energy crops like Panicum virgatum (switchgrass), Sorghum bicolor (sorghum, sudangrass), Miscanthus giganteus (miscanthus), Saccharum sp. (energycane), Populus balsamifera (poplar), Andropogon gerardii (big bluestem), Pennisetum purpureum (elephant grass), Phalaris arundinacea (reed canarygrass), Cynodon dactylon (bermudagrass), Festuca arundinacea (tall fescue), Spartina pectinata (prairie cord-grass), Medicago sativa (alfalfa), Arundo donax (giant reed), Secale cereale (rye), Salix spp. (willow), Eucalyptus spp. (eucalyptus), Triticosecale spp. (triticum-wheat X rye), and Bamboo; and starch-based energy crops like Zea mays (corn) and Manihot esculenta (cassaya); and sucrose-based energy crops like Saccharum sp. (sugarcane) and Beta vulgaris (sugarbeet); and biofuel-producing energy crops like Glycine max (soybean), Brassica napus (canola), Helianthus annuus (sunflower), Carthamus tinctorius (safflower), Jatropha curcas (Jatropha), Ricinus communis (castor), Elaeis guineensis (African oil palm), Elaeis oleifera (American oil palm), Cocos nucifera (coconut), Camelina sativa (wild flax), Pongamia pinnata (Pongam), Olea europaea (olive), Linum usitatissimum (flax), Crambe abyssinica (Abyssinian-kale), and Brassica juncea.

In certain preferred embodiments, the plant may be a coconut (Cocos nucifera), betel (Areca catechu), tea (Camellia sinensis), cocoa (Theobroma cacao), acacia (Acacia species), or poplar (Populus species) plant.

Treatment of Soil with Muscodor strobelii

In another aspect the invention provides methods for preventing or treating a plant-pathogen-related disease, in which the method includes applying a composition comprising one or more Muscodor strobelii organisms to soil or a plant growth medium. A non-soil plant growth medium can be sand, vermiculite, fibers, a gel or liquid based medium for plant growth, etc. In some embodiments, the soil or plant growth medium treated with a Muscodor strobelii composition contains seeds of plants of one or more plants of a species of interest. In some embodiments, soil or another plant growth medium is treated with a Muscodor strobelii composition and is subsequently planted with one or more plants of a species of interest. The planting can be planting of seeds, shoots, roots, or transplanting of whole plants (such as, but not limited to, seedlings). The M. strobelii composition can be mixed into the soil or growth medium, bored or injected into the soil or growth medium, sprayed, drenched, dusted, or scattered on the soil or growth medium, and optionally watered in.

The invention thus provides a method to use compositions of Muscodor strobelii to kill plant pathogens in soil. Suitable formulations for soil treatment are apparent to those of skill in the art and include wettable powders, granules, pellets, and the like, microencapsulations in a suitable medium and the like, liquids such as aqueous flowables and aqueous suspensions, and emulsifiable concentrates. Formulations may include food sources for the cultured organisms, such as barley, rice, or other organic materials such as empty fruit bunches.

For example, seeds, branches, mulch, plant fragments, and powdered milk may be useful to preserve M. strobelii viability for extended periods of time when mixed into soil. Chemical and biological pesticides may also be used to amplify the rate of Muscodor strobelii-mediated killing of plant pathogens, as well as to target other plant pathogens.

The invention includes a composition of isolated M. strobelii and an agricultural carrier. One agricultural carrier is soil itself. Other agricultural carriers include loam, clay, vermiculite, alginate, pellets made of mixtures of flour and inorganic materials, seeds, branches, mulch, plant fragments, and powdered milk. Muscodor strobelii may be formulated on a seed such as barley or rice or other waste plant materials, dried and then applied to the soil directly. Mixing may then be performed.

The composition may be turned into the soil prior to the planting of a crop or during the planting of seeds, roots, or shoots, or transplanting of plants, or the composition can be applied to the soil after plants have been established. For example, the composition with soil or an agricultural carrier may be applied to the base of oil palm plants. The composition may also be directly applied to the roots of the plants. The plants in some embodiments have root disease caused by a plant pathogen. The pathogen may be Ganoderma boninense. Other exemplary plant pathogens causing root diseases include Phytophthora palmivora, Pythium ultimum, or Sclerotinia sclerotiorum. It is contemplated that M. strobelii releases VOCs in the soil and/or in the vicinity of the plant that inhibit growth of or kill plant pathogenic fungi such as Ganoderma boninense, Phytophthora palmivora, Pythium ultimum, and Sclerotinia sclerotiorum.

In some embodiments, the invention provides for a method to pre-treat soil with M. strobelii, or alternatively, to inoculate soil with M. strobelii. The inoculated soil may be then used for in planting of seeds or plants susceptible to pathogens that can be effectively inhibited by M. strobelii. For example, M. strobelii in some embodiments is inoculated into the soil, optionally under conditions of being covered or sealed for several days, after which plants are placed in the soil with some reasonable certainty that no infestation will follow.

In certain preferred embodiments of the present invention, treatment of a plant growth medium such as soil with M. strobelii organisms results in a reduced occurrence of at least one plant disease caused by a bacterium, fungus, or insect as compared with plants grown in a medium not treated with an M. strobelii composition. In some embodiments, the plant disease is caused by Aspergillus fumigatus, Botrytis cinerea, Cerpospora betae, Curvularia spp, Ganoderma boninense, Geotrichum candidum, Mycosphaerella fijiensis, Phytophthora palmivora, Phytophthora ramorum, Pythium ultimum, Rhizoctonia solani, Rhizopus spp., Schizophyllum spp., Sclerotinia sclerotiorum, Verticillium dahliae, or Xanthomonas axonopodis. In a particularly preferred embodiment of the invention, the host plant is susceptible to diseases caused by Ganoderma boninense. In another preferred embodiment, the host plant is an oil palm plant. In other preferred embodiments, the plant may be a coconut (Cocos nucifera), betel (Areca catechu), tea (Camellia sinensis), cocoa (Theobroma cacao), acacia (Acacia species), or poplar (Populus species) plant. In some further preferred embodiments, the disclosed compositions and methods are effective to kill the plant pathogen.

In some embodiments, a method is provided for inhibiting or preventing developments of a plant pathogenic disease, in which a culture of M. strobelii, such as biologically pure M. strobelii, is grown in the vicinity of a host plant. The composition that includes isolated M. strobelii is applied to the plant or the plant growth medium, optionally in a composition that can include any of a stabilizer, a fungal food source, a wetting agent, or a dispersing agent, such as an emulsifier, powder, particulate, or surfactant. For example, before, concurrent with, or after planting of a plant species of interest, a composition of isolated M. strobelii can be applied to the plant or plant growth medium as a water-in-oil emulsion, with one or more ionic or nonionic surfactants, or mixed with any feasible material, such as but not limited to, flour, sugar, clay, vermiculite, sand, diatomaceous earth, silica, seed cases, ground barley or soybeans, in an extract broth of grain or legumes, agar, alginate, pellets of agar, clay, plant material, etc. The culture of M. strobelii can then grow in or on the plant, or in the plant growth medium surrounding the plant, preventing the development of a plant pathogenic disease. In some embodiments, establishing a culture of M. strobelii by applying a composition of isolated M. strobelii in the vicinity of a plant kills a plant pathogen that is associated with the plant or present in the growth medium of the plant.

In some embodiments, multiple applications of the M. strobelii composition are applied to the plants and/or soil surrounding the plants. The applications can be by the same means or different means (e.g., soil treatment followed by spraying the base of plants), and can use the same or different formulations of M. strobelii.

Another aspect of the invention provides a method for screening microbial strains that may be useful for treating, inhibiting or preventing the development of a plant pathogenic disease. The method involves (i) exposing or contacting candidate microbial strains with the invention composition, (ii) selecting microbial strains resistant to the composition, and (iii) characterizing the selected microbial strain. The characterization of the selected microbial strains can be carried by a variety of known molecular and microscopy techniques. Non-limiting examples of such techniques include electron microscopy, GC-MS analysis of VOC profile, PCR amplification and phylogenetic analysis of the 18S or the ITS-5.8S rDNA sequences.

### The Polynucleotides and Polypeptides of the Invention

In another aspect of the present invention, the disclosure provides novel substantially purified nucleic acid molecules, nucleic acid molecules that interfere with these nucleic acid molecules, nucleic acid molecules that hybridize to these nucleic acid molecules, and substantially purified nucleic acid molecules that encode the same protein due to the degeneracy of the DNA code. Additional embodiments of the present application further include the polypeptides encoded by the substantially purified nucleic acid molecules of the present invention.

The polypeptides and polypeptides of the present invention will preferably be “biologically active” with respect to either a structural attribute, such as the capacity of a nucleic acid to hybridize to another nucleic acid molecule, or the ability of a polypeptide to be bound by antibody (or to compete with another molecule for such binding). Alternatively, such an attribute may be catalytic and thus involve the capacity of the molecule to mediate a chemical reaction or response.

The polypeptides and polypeptides of the present invention may also be recombinant. As used herein, the term recombinant means any molecule (e.g. DNA, peptide etc.), that is, or results, however indirect, from human manipulation of a polynucleotide or polypeptide.

Nucleic acid molecules or fragment thereof of the present invention are capable of specifically hybridizing to other nucleic acid molecules under certain circumstances. As used herein, two nucleic acid molecules are said to be capable of specifically hybridizing to one another if the two molecules are capable of forming an anti-parallel, double-stranded nucleic acid structure. A nucleic acid molecule is said to be the “complement” of another nucleic acid molecule if they exhibit complete complementarity. As used herein, molecules are said to exhibit “complete complementarity” when every nucleotide of one of the molecules is complementary to a nucleotide of the other. Two molecules are said to be “minimally complementary” if they can hybridize to one another with sufficient stability to permit them to remain annealed to one another under at least conventional “low-stringency” conditions. Similarly, the molecules are said to be “complementary” if they can hybridize to one another with sufficient stability to permit them to remain annealed to one another under conventional “high-stringency” conditions. Conventional stringency conditions are described by Sambrook et al., In: Molecular Cloning, A Laboratory Manual, 2nd Edition, Cold Spring Harbor Press, Cold Spring Harbor, N.Y. (1989), and by Haymes et al. In: Nucleic Acid Hybridization, A Practical Approach, IRL Press, Washington, D.C. (1985). Departures from complete complementarity are therefore permissible, as long as such departures do not completely preclude the capacity of the molecules to form a double-stranded structure. Thus, in order for a nucleic acid molecule or fragment of the present invention to serve as a primer or probe it need only be sufficiently complementary in sequence to be able to form a stable double-stranded structure under the particular solvent and salt concentrations employed.

Appropriate stringency conditions which promote DNA hybridization are, for example, 6.0× sodium chloride/sodium citrate (SSC) at about 45° C., followed by a wash of 2.0×SSC at 50° C. The conditions are known to those skilled in the art, or can be found in Current Protocols in Molecular Biology, John Wiley & Sons, N.Y. (1989), 6.3.1-6.3.6. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature at about 22° C., to high stringency conditions at about 65° C. Both temperature and salt may be varied, or either the temperature or the salt concentration may be held constant while the other variable is changed.

In a preferred embodiment, a nucleic acid of the present invention will specifically hybridize to one or more of the nucleic acid molecules set forth in the Sequence Listing or complements thereof under moderately stringent conditions, for example, at about 2.0×SSC and about 65° C.

In a particularly preferred embodiment, a nucleic acid of the present invention will include those nucleic acid molecules that specifically hybridize to one or more of the nucleic acid molecules set forth in the Sequence Listing or complements thereof under high stringency conditions.

In another embodiment, the present invention provides nucleotide sequences comprising regions that encode polypeptides. The encoded polypeptides may be the complete protein encoded by the gene represented by the polynucleotide, or may be fragments of the encoded protein. Preferably, polynucleotides provided herein encode polypeptides constituting a substantial portion of the complete protein, and more preferentially, constituting a sufficient portion of the complete protein to provide the relevant biological activity.

Of particular interest are polynucleotides of the present invention that encode polypeptides involved in the production of VOCs, or in one or more important biological functions in plants. Such polynucleotides may be expressed in transgenic plants to produce plants having modulated phenotypic properties and/or modulated response to stressful environmental conditions.

A subset of the nucleic acid molecules of this invention includes fragments of the disclosed polynucleotides consisting of oligonucleotides of at least 15, preferably at least 16 or 17, more preferably at least 18 or 19, and even more preferably at least 20 or more, consecutive nucleotides. Such oligonucleotides are fragments of the larger molecules having a sequence selected from the polynucleotide sequences in the Sequence Listing, and find use, for example, as interfering molecules, probes and primers for detection of the polynucleotides of the present invention.

Also of interest in the present invention are variants of the polynucleotides provided herein. Such variants may be naturally occurring, including homologous polynucleotides from the same or a different species, or may be non-natural variants, for example polynucleotides synthesized using chemical synthesis methods, or generated using recombinant DNA techniques. With respect to nucleotide sequences, degeneracy of the genetic code provides the possibility to substitute at least one base of the protein encoding sequence of a gene with a different base without causing the amino acid sequence of the polypeptide produced from the gene to be changed. Hence, the DNA of the present invention may also have any base sequence that has been changed from any polynucleotide sequence in the Sequence Listing by substitution in accordance with degeneracy of the genetic code. References describing codon usage are readily publicly available.

Polynucleotides of the present invention that are variants of the polynucleotides provided herein will generally demonstrate significant identity with the polynucleotides provided herein. Of particular interest are polynucleotide homologs having at least about 50% sequence identity, at least about 60% sequence identity, at least about 70% sequence identity, at least about 80% sequence identity, at least about 85% sequence identity, and more preferably at least about 90%, 95% or even greater, such as 96%, 97%, 98% or 99% sequence identity with polynucleotide sequences described herein.

Nucleic acid molecules and fragments thereof of the present invention may be employed to obtain other nucleic acid molecules from the same species. Such nucleic acid molecules include the nucleic acid molecules that have the complete coding sequence of a protein and promoters and flanking sequences of such molecules. In addition, such nucleic acid molecules include nucleic acid molecules that encode for other isozymes or gene family members. Such molecules can be readily obtained by using the above-described nucleic acid molecules or fragments thereof to screen cDNA or genomic libraries obtained from Muscodor strobelii. Methods for forming such libraries are well known in the art.

Nucleic acid molecules and fragments thereof of the present invention may also be employed to obtain nucleic acid homologues. Such homologues include the nucleic acid molecules of different alleles within Muscodor species or other organisms, including the nucleic acid molecules that encode, in whole or in part, protein homologues of other organisms, sequences of genetic elements such as promoters and transcriptional regulatory elements. Such molecules can be readily obtained by using the above-described nucleic acid molecules or fragments thereof to screen cDNA or genomic libraries obtained from such plant species. Methods for forming such libraries are well known in the art. Such homologue molecules may differ in their nucleotide sequences from those found in one or more of the nucleotides in the Sequence Listing or complements thereof because complete complementarity is not needed for stable hybridization. The nucleic acid molecules of the present invention therefore also include molecules that, although capable of specifically hybridizing with the nucleic acid molecules may lack “complete complementarity.” In a particular embodiment, methods of 3′ or 5′ RACE may be used to obtain such sequences.

Any of a variety of methods known in the art may be used to obtain one or more of the above-described nucleic acid molecules. Automated nucleic acid synthesizers can be employed for this purpose. In lieu of such synthesis, the disclosed nucleic acid molecules can be used to define a pair of primers that can be used with the polymerase chain reaction to amplify and obtain any desired nucleic acid molecule or fragment, which is standard in the art.

The degeneracy of the genetic code, which allows different nucleotide sequences to code for the same protein or peptide, is further known in the art.

In an aspect of the present invention, one or more of the nucleic acid molecules of the present invention differ in nucleotide sequence from those encoding a Muscodor protein or fragment thereof selected from the group consisting of the nucleotide sequences in the Sequence Listing due to the degeneracy in the genetic code in that they encode the same protein but differ in nucleotide sequence.

In another further aspect of the present invention, one or more of the nucleic acid molecules of the present invention differ in nucleotide sequence from those encoding a Muscodor protein or fragment thereof selected from the group consisting of the nucleotide sequences in the Sequence Listing due to fact that the different nucleotide sequences encode a protein having one or more conservative amino acid residues. It is understood that genetic codons capable of coding for such conservative substitutions are well known in the art.

This invention also provides polypeptides that are encoded by the polynucleotides of the invention. It is known in the art that one or more amino acids in a sequence can be substituted with other amino acid(s), the charge and polarity of which are similar to that of the substituted amino acid, i.e. a conservative amino acid substitution, resulting in a biologically/functionally silent change. Conservative substitutes for an amino acid within the polypeptide sequence can be selected from other members of the class to which the amino acid belongs. Amino acids can be divided into the following four groups: (1) acidic (negatively charged) amino acids, such as aspartic acid and glutamic acid; (2) basic (positively charged) amino acids, such as arginine, histidine, and lysine; (3) neutral polar amino acids, such as serine, threonine, tyrosine, asparagine, and glutamine; and (4) neutral nonpolar (hydrophobic) amino acids such as glycine, alanine, leucine, isoleucine, valine, proline, phenylalanine, tryptophan, cysteine, and methionine.

Conservative amino acid changes within the native polypeptides' sequence can be made by substituting one amino acid within one of these groups with another amino acid within the same group. Biologically functional equivalents of the polypeptides or fragments thereof of the present invention can have about 10 or fewer conservative amino acid changes, more preferably about 7 or fewer conservative amino acid changes, and most preferably about 5 or fewer conservative amino acid changes. In a preferred embodiment of the present invention, the polypeptide has between about 5 and about 500 conservative changes, more preferably between about 10 and about 300 conservative changes, even more preferably between about 25 and about 150 conservative changes, and most preferably between about 5 and about 25 conservative changes or between 1 and about 5 conservative changes. The encoding nucleotide sequence will thus have corresponding base substitutions, permitting it to encode biologically functional equivalent forms of the proteins or fragments of the present invention.

Polypeptides of the present invention that are variants of the polypeptides provided herein will generally demonstrate significant identity with the polypeptides provided herein. Of particular interest are polypeptide homologs with at least about 50% sequence identity, at least about 60% sequence identity, at least about 70% sequence identity, at least about 80% sequence identity, at least about 85% sequence identity, and more preferably at least about 90%, 95% or even greater, such as 98% or 99% sequence identity with polynucleotide sequences described herein.

Any of a variety of methods well known in the art may be used to obtain one or more of the above-described polypeptides. The polypeptides of the invention can be chemically synthesized or alternatively, polypeptides can be made using standard recombinant techniques in heterologous expression systems such as E. coli, yeast, insects, etc.

### Information in the Sequence Listing

This specification contains nucleotide and polypeptide sequence information prepared using the program Patentln Version 3.5. Each sequence is identified in the Sequence Listing by the numeric indicator <210> followed by the sequence identifier. Sequences referred to in the specification are defined by the term “SEQ ID NO:”, followed by the sequence identifier (e.g. SEQ ID NO: 1 refers to the sequence in the Sequence Listing designated as <400>1). For each Muscodor gene, the EST contig with the longest overlap with the gene was included in the Sequence Listing as indicated by an EST identifier having the initial prefix “CUFF”.

The Muscodor sequences provided in the Sequence Listing are annotated to indicate one or several potential applications of the respective sequences. Some sequences are enzymes, i.e. catalysts of specific chemical or biochemical reactions, and their activity is indicated by enzyme classification (EC) numbers. The EC numbers used in the sequence listing correspond to the SWISSPROT enzyme classification system, as found for example at www.expasy.ch. Some sequences contain “pfam” domains which are indicative of particular applications. The specific pfam domains are described in more detail by various sources, such as www.sanger.ac.uk or pfam.janelia.org. Thus, various practical applications of the Muscodor sequences in the sequence listing are immediately apparent to those of skill in the art based on their similarity to known sequences.

Some Muscodor sequences in the Sequence Listing are annotated in the “miscellaneous features” section with valuable applications of the respective sequences in modulating the production of VOCs, such as enzymes involved in the pathways for the production of the VOCs. Thus, various practical applications of the Muscodor sequences in the Sequence Listing are immediately apparent to those of skill in the art based on their similarity to known sequences.

Expression characteristics of some Muscodor sequences in different growth conditions are indicated in the “miscellaneous features” fields for the respective sequences in the Sequence Listing. In most cases, expression characteristics are associated with the Muscodor sequences in the Sequence Listing by monitoring genome-wide gene expression using an internal Muscodor EST dataset. As those skilled in the art would readily appreciate, such expression data can be used as an indication of the potential for certain genes to play key roles in expression of different phenotypes. Moreover, it is a common practice of those skilled in the art to use such first-level genomic data to uncover sequences of interest and to derive a path toward identifying genes important in a particular pathway or response of interest. Differentially expressed sequences may be used in vectors for making transgenic organisms with modulated characteristics.

Additional information of sequence applications comes from similarity to sequences in public databases. Entries in the “miscellaneous features” sections of the Sequence Listing labeled “NCBI GI:” and “NCBI Desc:” provide additional information regarding the respective sequences. In some cases, the corresponding public records, which may be retrieved from www.ncbi.nlm.nih.gov, cite publications with data indicative of uses of the annotated sequences.

From the disclosure of the Sequence Listing, it can be seen that the nucleotides and polypeptides of the inventions are sometimes useful, depending upon the respective individual sequence, to make transgenic organisms with one or more altered characteristics. The present invention further encompasses nucleotides that encode the above described polypeptides, such as those included in the Sequence Listing, as well as the complements and/or fragments thereof, and include alternatives thereof based upon the degeneracy of the genetic code.

The nucleotide sequences according to the present invention further encompass those that encode appropriate proteins from any organism, in particular from plants, fungi, algae, bacteria or animals.

Exogenous genetic materials may be transferred into a cell and the cell regenerated into a whole transgenic organism. Techniques useful for transferring such genetic materials into either an endophytes or plant are well known in the art. The choice of promoters to be included depends upon several factors, including but not limited to efficiency, selectability, inducibility, desired expression level, and cell- or tissue-preferential expression. A large number of promoters which are active in plant cells or in endophyte cells have been described in the literature. One of skill in the art can routinely modulate the expression of a sequence by appropriately selecting and positioning promoters and other regulatory regions relative to the sequence.

Of particular interest in the present invention are polypeptides involved in one or more important biological properties in endophytes and plants, for example, the production of VOCs. Such polypeptides may be produced in transgenic plants or organisms that do not express such polypeptides, or may be modulated, such as over-expressed or up-regulated, in endophytes and organisms that already express such polypeptides to provide transgenic organisms having improved phenotypic properties and/or improved response to environmental conditions. Alternatively, decreased expression or down-regulation of such polypeptides may also be desired. Such decreased expression can be obtained by use of the polynucleotide sequences provided herein, for example in antisense or co-suppression methods. Invention polypeptides may be introduced alone or in combination with any additional polypeptides or compounds, chemical or biological, for example co-factors, substrates, stimulants, etc.

In certain embodiments, the molecules of the present invention may be introduced into the genome of a desired plant host by a variety of conventional transformation techniques, which are well known to those skilled in the art. Preferred methods of transformation of plant cells or tissues are the Agrobacterium mediated transformation method and the biolistics or particle-gun mediated transformation method. Suitable plant transformation vectors for the purpose of Agrobacterium mediated transformation include those derived from a Ti plasmid of Agrobacterium tumefaciens, as well as those disclosed, e.g., by Bevan (Nucleic Acids Res. 12: 8711-8721, 1984); Herrera-Estrella et al. (Nature 303:209, 1983); Klee et al. (Bio-Technology 3(7): 637-642, 1985). In addition to plant transformation vectors derived from the Ti or root-inducing (Ri) plasmids of Agrobacterium, alternative methods can be used to insert the DNA constructs of this invention into plant cells. Such methods may involve, but are not limited to, for example, the use of liposomes, electroporation, chemicals that increase free DNA uptake, free DNA delivery via microprojectile bombardment, and transformation using viruses or pollen.

In other preferred embodiments, the molecules of the present invention may be introduced into the genome of a desired endophyte, by a variety of transformation techniques, which are well known to those skilled in the art. (See for example, Panaccione et al., Proc Natl Acad Sci USA. 2001, 98(22), 12820-5).

The discussion of the general methods given herein is intended for illustrative purposes only. Other alternative methods and embodiments will be apparent to those skilled in the art upon review of this disclosure. The following examples are offered to illustrate, but not limit, the invention.

### Example 1

### Discovery of the Muscodor strobelii Fungus (Isolate Designated as MB-8 Herein)

A novel endophytic fungus termed Muscodor strobelii has been discovered in a philodendron plant south of Kuala Lumpur in the University of Malaysia forest at Bangi. This fungus is one of a group of endophytic fungi that belong to a fungal genus known as Muscodor. Members of the Muscodor fungal genus typically have a white mycelium with intertwining hyphae. These hyphae make rope-like strands and have never been observed to produce any fruiting structures, including spores, of any type. Muscodor species may produce volatile biologically active products that can affect other microbes, insects and nematodes. The newly isolated Muscodor strobelii produces a composition of volatile organic compounds that is different from that of all other Muscodor species that have been described to date.

Muscodor strobelii was isolated as follows. Several small stems of Philodendron sp were taken from a plant growing in the KL forest of the University of Malaysia in November of 2007. Several small (2-5 inch) pieces from the stems were cut and placed into 70% ethanol for 30 seconds under a laminar flow hood. A pair of sterile tweezers was used to hold the stems separately in the flame to remove excess alcohol. Then small pieces of inner tissue (beneath the bark) were excised and placed onto water agar. Once hyphae were observed, several hyphal tips were aseptically cut out of the agar and placed on fresh potato dextrose agar PDA. Isolate MB-8, comprising M. strobelii, was isolated in this manner. Several Petri plates of (PDA) were used to determine if the fungus produced volatile antibiotics. This procedure included removing a 1-inch section of the agar from the middle of the plate, plating a plug of the MB-8 isolate on one side and allowing it to grow for several days, and then plating test organisms on the other side of the gap (Strobel et al., 2001, Microbiology 147: 2943-2950). The isolate MB-8 demonstrated the ability to produce volatile antibiotics, which either inhibited or killed the fungi that were placed on the other side of the center well as test organisms, such as Pythium ultimum, Sclerotinia sclerotiorum.

### Example 2

### Scanning Electron Microscopy Characterization of Muscodor strobelii

Scanning electron microscopy was performed on isolate MB-8 after procedures described by Castillo et al., 2005, Scanning 27:305-311. Agar pieces and host plant pieces supporting fungal growth were placed in filter paper packets then placed in 2% glutaraldehyde in 0.1 M sodium cacodylate buffer (pH 7.2-7.4) with Triton X 100, a wetting agent, aspirated for 5 minutes and left overnight. The next day they were washed in six 15 minute changes in water buffer 1:1, followed by a 15 minute change in 10% ethanol, a 15 minute change in 30% ethanol, a 15 minute change in 50% ethanol, five 15 minute changes in 70% ethanol, and were then left overnight or longer in 70% ethanol. They were then rinsed six times for 15 minutes in 95% and then three 15 minute changes in 100% ethanol, followed by three 15 minute changes in acetone. The microbial material was critically point dried, gold sputtercoated, and images were recorded with an XL30 ESEM FEG in the high vacuum mode using the Everhart-Thornley detector. A 300× electron micrograph is provided as FIG. 1. Hyphae were measured using Image J software (available online on the World Wide Web at rsb.info.nih.gov/ij/).

### Example 3

### Growth and Storage of Muscodor strobelii

It was determined that MB-8 did not produce spores or any other fruiting bodies when several pieces of carnation leaves were placed on top of actively growing MB-8 to encourage spore production, and no such structures were observed after a week of incubation at 23° C. The fungus was also plated on several different media including Cellulose Agar (CA), Malt Agar (MA), and Corn Meal Agar (CMA) to determine if spore production of MB-8 would be displayed. With the exception of a slower growth rate on some of the media, no other characteristics of MB-8 appeared to be different, and no fruiting bodies or spores were observed.

Several methods were used to store the isolated fungus as a pure culture, one of which was the filter paper technique. The fungus was also allowed to grow on PDA, and then it was cut into small squares which were placed into vials containing 15% glycerol and stored at −70° C. The fungus was also stored at 4° C. by a similar method, using distilled water rather than glycerol. However, the most effective method of storage was on infested sterile barley seed at −70° C.

### Example 4

### Quantitative Analysis of Volatile Compounds from Muscodor strobelii

The gases in the air space above a fourteen day old culture of the MB-8 Muscodor strobelii growing on a Petri plate (PDA) were quantitatively analyzed. First, a baked “Solid Phase Micro Extraction” syringe (Supelco) consisting of 50/30 divinylbenzene/carburen on polydimethylsiloxane on a stable flex fiber was placed through a small hole drilled in the side of the Petri plate sporting the growth of B-23. The fiber was exposed to the vapor phase of the fungus for 45 minutes. The syringe was then inserted into the splitless injection port of a Hewlett Packard 6890 gas chromatograph containing a 30 m×0.25 mm I.D. ZB Wax capillary column with a film thickness of 0.50 mm. The column was temperature programmed as follows: 30° C. for 2 minutes followed to 220° C. at 5° C./min. The carrier gas was ultra high purity Helium (local distributor), and the initial column head pressure was 50 kPa. Prior to trapping the volatiles, the fiber was conditioned at 240° C. for 20 minutes under a flow of helium gas. A 30 second injection time was used to introduce the sample fiber into the GC. The gas chromatograph was interfaced to a Hewlett Packard 5973 mass selective detector (mass spectrometer) operating at unit resolution. Data acquisition and data processing were performed on the Hewlett Packard ChemStation software system. Initial identification of the unknowns produced by B-23 was made through library comparison using the NIST database. The identified compounds and their peak areas are provided in Table 1. A representative chromatogram is shown in FIG. 4.

TABLE 1
VOCs produced by a 14 day old culture of Muscodor strobelii on PDA.

| Retention | Peak | | |
|---|---|---|---|
| Time | Area | Possible Compound | MW |
| 3.853 | 6.07 | Acetone | 58 |
| 4.287 | 2.28 | 2-Butanone | 72 |
| 4.887 | 33.13 | Isobutyric acid, methyl ester | 102 |
| 5.682 | 122.88 | Isobutyric acid | 88 |
| 5.740 | 3.77 | Methyl 2-methoxypropenoate | 116 |
| 5.903 | 2.29 | Acetic acid, (tert-butylthio)- | 148 |
| 6.389 | 2.86 | 2-Methylheptanoic acid | 144 |
| 6.504 | 0.35 | Methyl 2,3-dimethylbutanoate | 130 |
| 6.961 | 11.04 | Allyl 2-methylpropanoate (isobutyric anhydride) | 128 |
| 7.512 | 1.13 | N,2-dimethylpropanamide | 101 |
| 9.090 | 0.39 | Heptyl allyl oxalate | 228 |
| 9.931 | 0.26 | Butyl propyl oxalate | 188 |
| 10.012 | 0.22 | 1-(ethenyloxy)-3-methyl-butane | 114 |
| 11.278 | 2.94 | alpha-Gurjunene | 204 |
| 11.332 | 2.15 | (-)-Aristolene | 204 |
| 11.436 | 1.07 | alpha-Patchoulene | 204 |
| 11.557 | 6.28 | 4,5-dimethyl-1,2,3,6,7,8,8a,8b- | 188 |
| | | octahydrobiphenylene | |
| 11.740 | 14.91 | (-)-tricyclo[6.2.1.0(4,11)]undec-5-ene,1,5,9,9- | 204 |
| | | tetramethyl-(isocaryophyllene-II) | |
| 11.929 | 0.44 | pentamethyl-benzene | 148 |
| 12.032 | 0.74 | Bergamotene | 204 |
| 12.099 | 0.14 | 4,4-diethyl-2,5-octadiyne | 162 |
| 12.190 | 0.34 | 2-(1-Propenyl)-6-methylphenol | 148 |
| 12.446 | 0.06 | (Z)-7,11-dimethyl-3-methylene-1,6,10- | 204 |
| | | dodecatriene | |
| 12.683 | 0.86 | 4,5-dehydro-isolongifolene | 202 |

### Example 5

### Fungal Cell Lysis and Acquiring 18 S, ITS-5.8S rDNA Sequence Information

A 2 week old culture of MB-8, growing on PDA, was used as a source of DNA after incubation at 25° C. MB-8 biomass was collected by brushing a pipet tip across the surface of the MB-8 mycelia. The biomass was transferred to a 100 μl PCR strip tube containing 50 μl of 100 mM Tris, pH 8.0. The biomass was homogenized via repeated up-and-down pipeting. A 2 μl aliquot of the MB-8 homogenate was then mixed with 2 μl of a 2× lysis buffer consisting of 100 mM Tris HCL, pH 8.0, 2 mM EDTA, 1% SDS, and 20 μl/ml Proteinase K (Goldenberger et al., 1995, PCR Methods Appl. 4:368-370). The lysis reaction was performed in a PTC-200 personal thermocycler (MJ-Research, MA, USA) as follows: 55° C. for 60 minutes, followed by 10 minutes at 95° C. A 2 μl aliquot of the lysis product was used as the source of template DNA for PCR amplification. The ITS1, 5.8S ITS2 rDNA sequence was amplified via PCR using the primers ITS1 (TCCGTAGGTGAACCTGCGG; SEQ ID NO:40286) and ITS4 (TCCTCCGCTTATTGATATGC; SEQ ID NO:40287) provided in the Sequence Listing.

The PCR mixture was prepared in a volume of 50 μl and consisted of 2 μl DNA from the fungal lysis reaction, 0.5 μl primer ITS1 and 0.5 μl primer ITS4, 5 μl 10% Tween-20 and 42 μl of Platinum PCR SuperMix (Invitrogen, CA, USA). The PCR was carried out in a PTC-200 personal thermocycler (MJ-Research, MA, USA) as follows: 94° C. for 10 minutes followed by 30 cycles of 94° C. for 30 seconds, 52° C. for 30 seconds and 72° C. for 1 minute, 15 seconds, followed by a 72° C. cycle for 10 minutes. A 10 μl aliquot of PCR product diluted in 10 μl of ddH₂O was run on a pre-cast 1.0% agarose E-Gel 96 with ethidium bromide (Invitrogen, CA, USA) for 10 minutes using the Mother E-Base (Invitrogen, CA, USA) as a power source. A gel image was obtained by UV fluorescent imaging using the Chemilmager Ready system (Alpha Innotech, CA, USA). The presence of a 400-500 bp PCR product was confirmed by the gel image. The remaining 40 μl of PCR product was cleaned using 6 μl of the ExoSAP-IT clean-up mix (USB, OH, USA). The purification reaction was run on a PTC-200 personal thermocycler (MJ-Research, MA, USA): 30 minutes at 37° C. followed by 30 minutes at 80° C. Purified products were frozen and submitted for PCR sequencing. Sequencing was performed in the forward and reverse priming directions using ITS1 and ITS4 primers by the J. Craig Venter Institute in San Diego, Calif. using 454 technologies.

Sequence of the 18s intergenic region (ITS) and partial sequence of the 18s rDNA are provided in the Sequence Listing as SEQ ID NO:40284 and SEQ ID NO:40285, respectively. The sequences were submitted to GenBank on the NCBI web site. Sequences obtained in this study were compared to the GenBank database using the BLAST software on the NCBI web site.

### Example 6

### Phylogenetic Reconstructions

Nucleotide sequences were aligned in Bioedit (located on the World Wide Web) followed by manual refinement. Phylogenetic trees were constructed in PHYML (located on the World Wide Web) using maximum likelihood, HKY substitution model and the default settings. Branch support was obtained by bootstrapping (100 replicates). A phylogenetic tree showing the relatedness of M. strobelii to other fungal species is shown in FIG. 3.

### Example 7

### Use of Muscodor strobelii to Kill Ganoderma boninense and Other Pathogens

Muscodor strobelii was grown on a half plate of PDA for 6 days (middle 2 cm of agar removed). Then a small block of agar (3×3×3 mm) containing the test organism was placed on the opposite side of the plate. The plate was sealed with parafilm and growth of the test organism was observed after two days, after which the test organisms were removed and placed on fresh plates of PDA to determine if they were alive or dead. The results demonstrated that the VOCs of M. strobelii were lethal to many plant pathogens including Ganoderma boninense and Phytophthora palmivora (see Table 2). The results indicate that Muscodor strobelii and its VOCs can be useful in controlling major diseases of plants, such as the basal stem rot disease of oil palm caused Ganoderma boninense. A comparable test using Muscodor albus against Ganoderma boninense resulted in no effect of its VOCs on Ganoderma boninense. Thus, also in these respects M. strobelii is biologically different from M. albus.

In a separate experiment, the same test was used to determine the effect of exposure to M. strobelii VOCs on Candida albicans, Escherichia coli, and Staphylococcus aureus. In each case, growth of the microorganisms was inhibited and subsequent growth on fresh plates was not observed indicating they were killed by exposure to the VOCs.

TABLE 2
Inhibition of growth/killing of microorganisms by Muscodor strobelii.

| Microorganism tested | Growth after 2 days exposure | Alive or Dead |
|---|---|---|
| Ganoderma boninense | No | Dead |
| Phytophthora palmivoria | No | Dead |
| Pythium ultimum | No | Dead |
| Botrytis cinerea | No | Dead |
| Geotrichum candidum | No | Dead |
| Verticillium dahaliae | No | Dead |
| Sclerotinia sclerotiorum | No | Dead |
| Trichoderma virdae | No | Dead |
| Ceratocystis ulmi | No | Dead |
| Cerpospora betae | No | Dead |
| Mycosphaerella fijiensis | No | Dead |
| Aspergillis fumigatus | No | Dead |
| Fusarium oxysporum | Yes | Alive |
| Fusarium solani | Yes | Alive |
| Muscodor albus* | Yes | Alive |
| Candida albicans | No | Dead |
| Escherichia coli | No | Dead |
| Staphylococcus aureus | No | Dead |

### Example 8

Protection of Oil Palm Plant Roots from Ganoderma boninense Infection Using Muscodor strobelii

Muscodor strobelii was tested to determine if it could protect oil palm roots from infection by Ganoderma boninense. To this end a root model experiment was set up and it employed 5 ml plastic tubes containing 5 ml of Malaysian sandy loam soil (wetted) under the following conditions: Tube 1 Soil only; Tube 2 soil with Ganoderma boninense inoculum (0.5 mg dry weight inoculum) administered as diced agar plugs 5 plugs (1 cm squared); Tube 3 contained 10 barley seeds with M. strobelii and Tube 4 contained both Ganoderma boninense and the barley seed M. strobelii inoculum. Then, into each tube was placed a small steel rod which opened up a hole into which a freshly cut small root of oil palm could be placed. The root itself was wiped with a Kimwipe that had been wetted with 70% alcohol. The soil was gently pushed into place around the root. The tubes were each given 50 μl of water, capped and incubated for 6 days. At the end of this time the roots were examined for disease symptoms and only the root having the Ganoderma boninense alone (Tube 2) showed any signs of infection, at one location on the root (see FIG. 3). The other roots all appeared healthy. The results show that M. strobelii can protect oil palm roots from Ganoderma boninense infection.

Another experiment of the same design used Phytophthora palmivora as the potential root infecting pathogen. Again, the results showed that M. strobelii acted to protect the oil palm roots from infection.

### Example 9

### Use of Muscodor strobelii to Kill Ganoderma boninense in Soil

Malaysian sandy loam soil was acquired from an oil palm plantation, and 25 ml of the sterilized soil was placed in 4 plastic centrifuge tubes. A set of holes was drilled into each tube in order to access the soil for sampling purposes. Into Tube 1 was placed only soil as a control. Into Tube 2 was placed 10 seeds of barley grains (infested with Muscodor strobelii). Into Tube 3 was placed the equivalent of 1.0 mg dry weight of Ganoderma boninense however it was in the form of diced PDA agar pieces (10 pieces 1 cm square surface area). Finally, into Tube 4 was placed the Ganoderma boninense inoculum and the 10 infested barley grains of M. strobelii. The tubes were capped and incubated at 23° C. for 3 days.

At the end of three days, a SPME fiber was inserted into one of the access holes of each tube and positioned there for at least 30-40 minutes. Then, the sample was run by GC/MS. The results show that it was possible to detect isobutyric acid, the major signature compound of M. strobelii, only in the soil samples containing the barley grains carrying M. strobelii. The gas chromatograph depicted in FIG. 4 includes tracings for all four of the tubes. Only compounds mixtures from Tubes 2 and 4 (the upper two tracings in the figure) show a peak at 4.45 minutes that corresponds to isobutyric acid.

We then attempted to isolate Ganoderma boninense from all of the soil samples by plating them on PDA. Ganoderma boninense could only be successfully isolated from the control Tube 3 that contained only Ganoderma boninense. Attempts were made to isolate M. strobelii from all of the tubes by the soil plating technique, and it was successfully isolated from Tube 2 and Tube 4. Confirmation of the identity of the two fungi was done by 18S rDNA and ITS sequence analysis (see Table 3).

The results show that M. strobelii kills all hyphae of Ganoderma boninense when grown together in soil, as it was not possible to recover Ganoderma boninense in soil that had been inoculated with M. strobelii. Thus, M. strobelii can control Ganoderma boninense in the soil.

TABLE 3
Molecular biological analysis to identify Ganoderma and Muscodor
isolates obtained from soil sample tubes 2, 3 and 4.

| | TUBE | |
|---|---|---|
| Query | Number | Description E-value |
| Gan1_18S | Tube 3 | Ganoderma boninense 18S small subunit |
| | | ribosomal RNA gene, partial sequence |
| Gan1_ITS | | |
| Gan2_18S | | Ganoderma boninense 18S small subunit |
| | | ribosomal RNA gene, partial sequence |
| Gan2_ITS | | Ganoderma sp. STK-2006a internal transcribed |
| | | spacer 1 (isolate 2) |
| MB8_1_18S | Tube 4 | Muscodor strobelii 18S ribosomal RNA gene, |
| | | partial sequence |
| MB8_1_ITS | | Fungal endophyte isolate 2161 18S ribosomal |
| | | RNA gene |
| MB8_2_18S | | Muscodor strobelii 18S ribosomal RNA gene, |
| | | partial sequence |
| MB8_2_ITS | | Fungal endophyte isolate 2161 18S ribosomal |
| | | RNA gene |
| 130_1_18S | Tube 2 | Muscodor strobelii 18S ribosomal RNA gene, |
| | | partial sequence |
| 130_1_ITS | | Fungal endophyte isolate 2161 18S ribosomal |
| | | RNA gene |

### Example 10

### A Muscodor strobelii Inoculum Formulation for Application in Agricultural Systems

Seeds of an appropriate grain such as barley or rice were mixed with an equal weight of empty fruit bunches (EFBs) that had been chopped into small pieces (0.1-3.0 cm) and sterilized by autoclaving. Water was added to the mixture to provide a growth medium for the fungus. Muscodor strobelii was first grown on potato broth for at least 7-10 days and the resulting culture was used as the inoculum for the grain/fruit bunch organic mass (depicted in FIG. 5). Incubation was under sterile conditions for 7-10 days. Once the fungus has covered the organic mass, the organic mass (grain/empty fruit bunches covered with fungus) was subjected to drying under atmospheric conditions. Powdered milk can be used to stabilize the viability of the fungus in the dried mass.

The infested mass was stored at room temperature, under refrigeration (approximately 4° C.), at freezer temperatures (approximately −20° C.), or at −70° C. Storage at −70° C. resulted in the fungus maintaining viability for a longer time than storage at other temperatures. The EFB cultures lost viability gradually at room temperature until after 3 months the viability of the stored culture was unreliable.

### Example 11

### Genomic DNA sequencing

A fresh culture of Muscodor strobelii MB8 was prepared for genomic DNA extraction. 4.3 g cell pellet was used for high molecular weight DNA extraction using the UltraClean® Mega Soil DNA Isolation Kit (Cat. No 12900-10) from MO BIO Laboratories, Inc according to the manufacture's recommended protocol.

The genomic DNA from Muscodor strobelii MB8 was prepared for shotgun 454-pyrosequencing. Genomic DNA (7.5 μg) was used for library construction according to the recommended protocol (454 Life Sciences) for single long reads. The sequences were generated by two GS FLX Titanium series sequencing runs.

### Example 12

### cDNA Sequencing

Cell cultures and mRNA isolation: MB8 fungal cultures were grown in potato dextrose broth liquid medium or potato dextrose agar solid medium for several days until dense mycelia had filled the medium. The fungus was harvested from liquid medium by centrifugation at 10,000×g for 10 minutes, and from solid medium by scraping the mycelium off with a spatula. The fungal tissue was scooped up with a spatula and frozen in liquid nitrogen in pea-sized pieces. Equal amounts of the frozen tissue pieces were weighed into 50 ml polycarbonate tubes chilled in liquid nitrogen.

Stainless steel balls were added to each polycarbonate tube containing fungal tissue. The frozen tissue was pulverized by shaking in a Spex GenoGrinder™ tube shaker. The pulverized tissue was transferred to 5 parts lysis buffer (100 mM Tris base, pH 9.0-9.5, 250 mM LiCl, 50 mM EDTA, 10% SDS, 1.5% β-mercaptoethanol) and suspended/thawed completely in the buffer. The suspension was extracted with an equal volume of chloroform, centrifuged, and the supernatant mixed with an equal volume of RNA stabilization buffer (45% w/v guanidine sulfate, 25 mM sodium citrate, 0.5% sodium lauryl-sarcosine, 1 M sodium acetate pH 5.0). The mixture was allowed to stand for 15 minutes at room temperature and was then extracted with ¼ volume of chloroform and centrifuged. Total RNA was isolated from the resulting supernatant using Qiagen RNeasy Maxi™ columns used according to the manufacturer's recommendations. The total RNA was eluted in 2 ml RNAse-free distilled water, quantitated, and precipitated in isopropanol. The precipitated RNA was dissolved in Tris-EDTA buffer at a concentration of 5 mg/ml and mRNA purified from the total RNA using Invitrogen's FastTrack MAG TM mRNA isolation kit used according to the manufacturer's recommendations. The mRNA was eluted with 200 μl distilled water and stored at −80° C.

cDNA synthesis and sequencing: cDNA pools derived from fungal tissue grown in solid and liquid medium (VOC-producing conditions) were generated and sequenced separately. cDNA was synthesized by fragmenting the RNA and converting it to cDNA with random primers using the Illumina mRNA-Seq Library Preparation Kit according to the manufacturer's recommendation. Illumina adapters were then ligated to the DNA ends and the sample was PCR amplified using reagents in the same kit. The DNA template was sequenced on an Illumina Genome Analyzer II platform according to the manufacturer's recommended conditions. 76 bp paired-end reads were generated and mapped to the assembled genome sequence.

### Example 13

### Sequence Assembly and Gene Identification

MB8 genome sequence assembly was carried out using Newbler assembler version 2.0.00.20, using default parameters except the minimum overlap identity parameter (-mi), which was increased from 90% to 93%. The inputs consisted of 2 plates of 454 FLX Titanium pyrosequencing (unpaired) reads. In total, the assembler used 889 Mbp of input bases in 2.36 M reads. The assembled result incorporated about 99% of the reads and provided 16.4× coverage on average. The assembly size was bounded by 53.6 Mbp (total bases in contigs). The contig N50 value was 141.7 Kbp, and the largest contig was 631.4 Kbp.

A training set of 2528 high confidence gene models supported by several lines of evidence was generated using the following proprietary eukaryotic gene prediction procedure. UniProt protein sequences (release 15.9, Apweiler et al., Nucleic Acids Res. 32:D115-D119, 2004) from the Kingdom Fungi were aligned to the assembled MB8 genome using Blastx (Altschul et al., Nucleic Acids Res. 25: 3389-3402, 1997). Precise intron/exon boundaries for protein sequences that had good blast hits to the genome were then predicted using GeneWise (Birney and Durbin, Genome Res. 10: 547-548, 2000). All protein-DNA alignments were filtered to remove sequences with frameshifts, inner stop codons, and sequences without a valid start or stop codon at the 5′ and 3′ ends respectively. Additional filtering was performed to include only gene models with multiple lines of supporting experimental evidence. These included 1) the presence of splice junctions obtained by aligning Solexa/Illumina paired-end cDNA reads to the MB8 genome using the program TopHat (Trapnell et al., Bioinformatics 25: 1105-1111, 2009), and 2) at least 50% overlap between the gene model and the corresponding transcript assembled from the Solexa/Illumina reads using the program Cufflinks (cufflinks.cbcb.umd.edu/). The resulting subset of gene models was thus supported both by full length protein-DNA alignments and by Solexa/Illumina cDNA sequences. The remaining training set gene models were further filtered to prevent over-fitting by removing 1) additional copies of gene models that were more than 55% identical at the protein level and 2) a subset of gene models with 2 or less introns in order to reduce the bias in the training gene set towards 1 or 2 exon genes. These steps ensured a more evenly distributed representative set of gene models. The final set of filtered gene model sequences was used to train a Hidden Markov Model (HMM) using the program Augustus (Starke et al., BMC Bioinformatics 7, 2006).

Genes were predicted ab initio on all of the assembled scaffold sequences using the HMM trained using Augustus. In addition to the HMM-based ab initio gene model, further direct evidence on gene structure was included in the predictions using the hints mechanism included in the Augustus program. This mechanism allows providing additional evidence on gene features such as exon-intron boundaries that Augustus can use to determine for example the location of an exon-intron boundary that is both consistent with the ab initio model and is supported by direct experimental data. The evidence used in MB8 gene finding included GeneWise protein-DNA alignments, Solexa based exon-intron splice junctions generated using Tophat, and assembled transcripts created using the program Cufflinks with Solexa reads. The weights for all hints were derived by optimizing them using an accuracy function based on the sensitivity and specificity of gene prediction results on Arabidopsis genome sequence using the manually curated Arabidopsis genome annotation (TAIR database, www.arabidopsis.org/) as a reference data set. Alternative transcripts for genes were also predicted when the evidence supported their presence.

The ESTs were also clustered to provide an estimate of the total number of protein encoding genes in the MB8 genome and to enable the extraction of as much information about each transcript as possible. Solexa/Illumina paired-end shotgun cDNA sequences were aligned to the assembled MB8 genome sequence using TopHat (Trapnell et al., Bioinformatics 25(9):1105-1111, 2009). The aligned sequences were then assembled into EST contigs using Cufflinks (from cufflinks.cbcb.umd.edu). Expression information using the approach above was obtained for predicted genes 1) from cells grown under volatile producing conditions 2) from cells grown under conditions where volatile compounds were not produced.

A number of embodiments of the invention have been described. Nevertheless, it will be understood that elements of the embodiments described herein can be combined to make additional embodiments and various modifications may be made without departing from the spirit and scope of the invention. Accordingly, other embodiments, alternatives and equivalents are within the scope of the invention and claimed herein. Headings within the applications are solely for the convenience of the reader, and do not limit in any way the scope of the invention or its embodiments.

All publications and patent applications mentioned in this specification are herein incorporated by reference to the same extent as if each individual publication or patent application was specifically can individually indicated to be incorporated by reference

What is claimed is:

1. An isolated culture of a strain of Muscodor strobelii.

2. An isolated strain of Muscodor strobelii, wherein the strain is Agricultural Research Service Culture Collection accession number NRRL 50288.

3. A culture of Muscodor strobelii according to claim 1, wherein the strain is capable of producing isobutyric acid or a derivative of any thereof.

4. A culture of Muscodor strobelii according to claim 1, wherein the strain is capable of producing one or more of aristolene, bergamotene, caryophyllene, gurjunene, isolongifolene, patchoulene, or a derivative of any thereof.

5. A culture of Muscodor strobelii according to claim 1, further comprising an agriculturally effective amount of a compound or composition selected from the group consisting of a bactericide, a fungicide, an insecticide, a microbicide, a nematicide, a fertilizer, and a food preservative.

6. A composition comprising a culture of Muscodor strobelii according to claim 1 and a carrier.

7. A composition according to claim 6, wherein the carrier is a seed.

8. A composition according to claim 6, wherein the composition is in the form of a powder, a granule, a pellet, a gel, an aqueous suspension, a solution, or an emulsion.

9. A method for treating, inhibiting or preventing the development of a plant pathogenic disease, said method comprising applying a culture of Muscodor strobelii according to claim 1 on or in the vicinity of a host plant.

10. A method according to claim 9, wherein the pathogen is selected from the group consisting of Aspergillus fumigatus, Botrytis cinerea, Cerpospora betae, Curvularia sp., Ganoderma boninense, Geotrichum candidum, Mycosphaerella filiensis, Phytophthora palmivora, Phytophthora ramorum, Pythium ultimum, Rhizoctonia solani, Rhizopus sp., Schizophyllum sp., Sclerotinia sclerotiorum, Verticillium dahliae, and Xanthomonas axonopodis.

11. A method according to claim 9, wherein the host plant is susceptible to Ganoderma boninense.

12. A method according to claim 9, wherein the host plant is an oil palm plant.

13. A method according to claim 9, wherein Muscodor strobelii is established as an endophyte on the plant.

14. A non-naturally occurring oil palm cultivar that is an oil palm infected with an isolated culture of Muscodor strobelii.

15. Seed, reproductive tissue, vegetative tissue, plant parts, or progeny of a non-naturally occurring oil palm cultivar according to claim 14.

16. A product comprising material derived from a non-naturally occurring cultivar according to claim 14.

17. A method for killing a plant pathogen, said method comprising growing a culture of Muscodor strobelii according to claim 1 in the vicinity of the plant pathogen.

18. A method for treating, inhibiting or preventing the development of a plant pathogenic disease, said method comprising growing a culture of Muscodor strobelii according to claim 1 in a growth medium or soil of a host plant prior to or concurrent with host plant growth in said growth medium or soil.

19. A method of screening microbial strains, said method comprising (i) co-culturing a Muscodor strobelii strain according to claim 1 with one or more candidate microbial strains, (ii) selecting one or more viable microbial strains after the co-culturing, and (iii) characterizing the one or more selected microbial strains; wherein said method identifies one or more microbial strains useful for treating, inhibiting or preventing the development of a plant pathogenic disease.

20. An isolated microbial strain obtainable by a method according to claim 19.

21. A method for killing, inhibiting or preventing the development of an organism selected from the group consisting of a fungus, a bacterium, a microorganism, a nematode, and an insect, said method comprising exposing the organism to an effective amount of a culture of Muscodor strobelii according to claim 1.

22. A substantially purified nucleic acid molecule comprising:
(i) a nucleotide sequence hybridizing under high stringency conditions to any one of the nucleotide sequences in the Sequence Listing, a complement thereof or a fragment of either;
(ii) a nucleotide sequence exhibiting a 70% or greater identity to any one of the nucleotide sequences in the Sequence Listing, a complement thereof or a fragment of either;
(iii) a nucleotide sequence encoding an amino acid sequence exhibiting a 50% or greater identity to any one of the polypeptides in the Sequence Listing; or
(iv) a nucleotide sequence that is an interfering RNA to a nucleotide sequence according to any one of paragraphs (i)-(iii).

23. A substantially purified polypeptide, wherein said polypeptide is encoded by a nucleic acid molecule comprising:
(i) a nucleotide sequence hybridizing under high stringency conditions to any one of the nucleotide sequences in the Sequence Listing, a complement thereof or a fragment of either;
(ii) a nucleotide sequence exhibiting a 70% or greater identity to any one of the nucleotide sequences in the Sequence Listing, a complement thereof or a fragment of either; or
(iii) a nucleotide sequence encoding an amino acid sequence exhibiting a 50% or greater identity to any one of the polypeptides in the Sequence Listing.

24. A transformed cell comprising a first nucleic acid molecule according to claim 22, wherein said first nucleic acid molecule is operably linked to a second nucleic acid molecule that is heterologous with respect to said first nucleic acid.

25. A transgenic organism comprising the transformed cell of claim 24.

26. The transgenic organism of claim 25, wherein said organism is an endophyte or a plant.
